(19) United States
(12) Patent Application Publication
(10) Pub. No.: US 2002/0107385 A1
(43) Pub. Date: Aug. 8, 2002

(54) Human breast tumor-specific proteins

(75) Inventors: Ingrid E. Akerblom, Redwood City, CA (US); Jennifer L. Hillman, San Jose, CA (US); Lynn E. Murry, Portola Valley, CA (US); Surya K. Goli, Sunnyvale, CA (US); Phillip R. Hawkins, Mountain View, CA (US)

Correspondence Address: INCYTE GENOMICS, INC., PATENT DEPARTMENT, 3160 Porter Drive, Palo Alto, CA, 94304, US

(73) Assignee: Incyte Pharmaceuticals, Inc.

(21) Appl. No.: 09/934,054

(22) Filed: Aug. 21, 2001

Related U.S. Application Data
(63) Continuation of application No. 08/747,547, filed on Nov. 12, 1996, now abandoned.

(51) Int. Cl. A01K 67/00; C07H 21/04; C12N 1/21; C07K 14/705; C12P 21/02; C12N 5/06

(52) U.S. Cl. 536/23.5; 435/69.1; 530/350; 435/325; 435/320.1; 435/252.3; 800/8

(57) ABSTRACT

The present invention provides polynucleotides that identify and encode two human steroid binding proteins (hSBP). The invention provides for genetically engineered expression vectors and host cells comprising the nucleic acid sequences encoding hSBP polypeptides. The invention also provides for the use of substantially purified hSBP polypeptides, antagonists, and nucleotide sequences (e.g., antisense sequences) in pharmaceutical compositions for the treatment of diseases associated with the expression of hSBP, specifically in the treatment of breast cancer. The invention also describes diagnostic assays for the detection of breast cancer in a susceptible or affected patient. The diagnostic assays utilize compositions comprising the polynucleotides encoding hSBP polypeptides or the complements thereof, which hybridize with the genomic sequence or the transcript of polynucleotides encoding hSBP or anti-hSBP antibodies that specifically bind to an hSBP polypeptide.

This application is a continuation application of U.S. application Ser. No. 08/747,547, filed Nov. 12, 1996, entitled HUMAN BREAST TUMOR-SPECIFIC PROTEINS, all of which applications and patents are hereby incorporated herein by reference.

### FIELD OF THE INVENTION

The present invention relates to nucleic acid and amino acid sequences of proteins that are differentially expressed in human breast tumor cells and to the use of these sequences in the diagnosis, study, prevention and treatment of disease.

### BACKGROUND OF THE INVENTION

Development of breast cancer is associated with multiple genetic changes associated with alterations in expression of specific genes. Breast cancer tissues express genes that are not expressed, or expressed at lower levels, by normal breast tissue. Thus, it is possible to differentiate between normal (non-cancerous) breast tissue and cancerous breast tissue by analyzing differential gene expression between tissues. In addition, there may be several possible alterations that lead to the various possible types of breast cancer. Thus, different types of breast tumors (e.g., invasive vs. non-invasive, ductal vs. axillary lymph node) can be differentiable one from another by the identification of the differences in genes expressed by different types of breast tumor tissues (Porter-Jordan et al. 1994 Hematol Oncol Clin North Am 8:73-100). Breast cancer can thus be generally diagnosed by detection of expression of a gene or genes associated with breast tumor tissue. Where enough information is available about the differential gene expression between various types of breast tumor tissues, the specific type of breast tumor can also be diagnosed.

Nucleotide and amino acid sequences associated with breast tumors can serve as genetic markers of inheritable breast cancer. Genetic changes on chromosome 17 are the most frequently identified events associated with breast tumors. At least four markers on chromosome 17 have been identified: p53 on 17p13.1, regions of loss of heterozygosity (LOH) on 17p13.3 and 17q12-qter, the breast/ovarian cancer locus (BRCA-1) on 17q21, and a fourth breast cancer growth suppressor gene on chromosome 17 (Casey et al. 1993 Hum Molec Genet 2:1921-1927).

Such genetic markers can also be useful in identifying patients susceptible to breast cancer. For example, the genetic marker BRCA-1 has been linked to a susceptibility of developing breast and/or ovarian cancer at a young age in a number of families (Hall et al. 1990 Science 250:1684-1689; Solomon et al. 1991 Cytogenet Cell Genet 58:686-738). The cumulative risks of developing breast cancer associated with the BRCA-1 marker are 50% at 50 years and 82% at 70 years (Easton et al. 1993 Am J Hum Genet 52:678-701). However, since the gene encoding BRCA-1 has not been cloned or sequenced, identification of an individual carrier of BRCA-1 is not possible without use of linkage analysis. Linkage analysis is generally not feasible in clinical practice since the genetic epidemiology required is tedious, if not impossible, in most cases (Kent et al. 1995 Europ J Surg Oncol 21:240-241).

The discovery of nucleotide sequences and polypeptides encoding proteins associated with breast cancer would satisfy a need in the art by providing new means of diagnosing and treating breast cancer.

### SUMMARY

The present invention features two human steroid binding proteins (hereinafter referred to individually as hSBP1, and hSBP2, and collectively as hSBP), and the full-length nucleotide sequences encoding these proteins, which are differentially expressed in human breast tumor tissue. The transcripts encoding these proteins are present in breast tumor tissue. The first polypeptide, referred to hereinafter as human steroid binding protein C1 (hSBP1), is characterized as having amino acid sequence homology to rat prostatic binding proteins C1 and C2 (PSC1₁₃RAT and PSC2_RAT′ respectively) and nucleotide sequence homology to hamster FHG 22 (GI 206441). The second polypeptide, referred to hereinafter as human steroid binding protein C2 (hSBP2), is characterized as having identity to human mammaglobin and homology to rat prostatic binding protein C3 (GI 206448). Accordingly, the invention features two substantially purified human steroid binding proteins, as shown in amino acid sequences of SEQ ID NO:1 and SEQ ID NO:3.

One aspect of the invention features isolated and substantially purified polynucleotides that encode hSBP. In a particular aspect, the polynucleotide is the nucleotide sequence of SEQ ID NO:2 and SEQ ID NO:4. In addition, the invention features polynucleotide sequences that hybridize under stringent conditions to SEQ ID NO:2 and SEQ ID NO:4.

The invention additionally features nucleic acid sequences encoding hSBP polypeptides, oligonucleotides, peptide nucleic acids (PNA), fragments, portions or antisense molecules thereof, and expression vectors and host cells comprising polynucleotides that encode hSBP. The present invention also relates to antibodies which bind specifically to an hSBP polypeptide, pharmaceutical compositions comprising substantially purified hSBP, fragments thereof, or antagonists of hSBP, in conjunction with a suitable pharmaceutical carrier, and methods for producing hSBP.

### BRIEF DESCRIPTION OF THE FIGS.

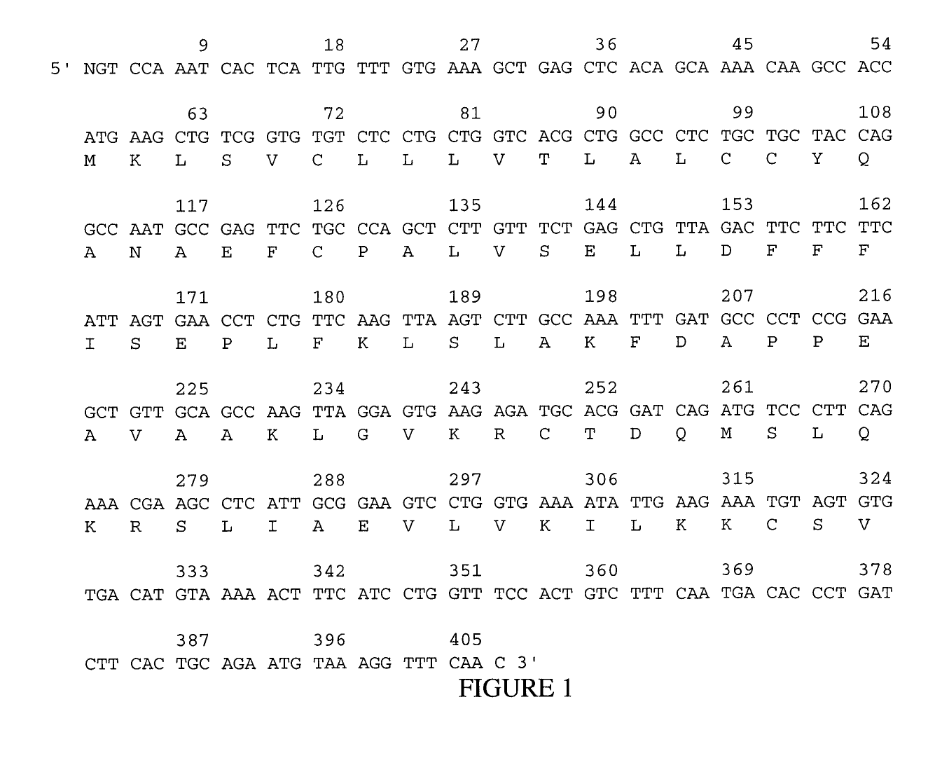

FIG. 1 shows the amino acid sequence (SEQ ID NO:1) and nucleic acid sequence (SEQ ID NO:2) of human steroid binding protein C1, hSBP1. The alignment was produced using MacDNAsis software (Hitachi Software Engineering Co Ltd, San Bruno, Calif.).

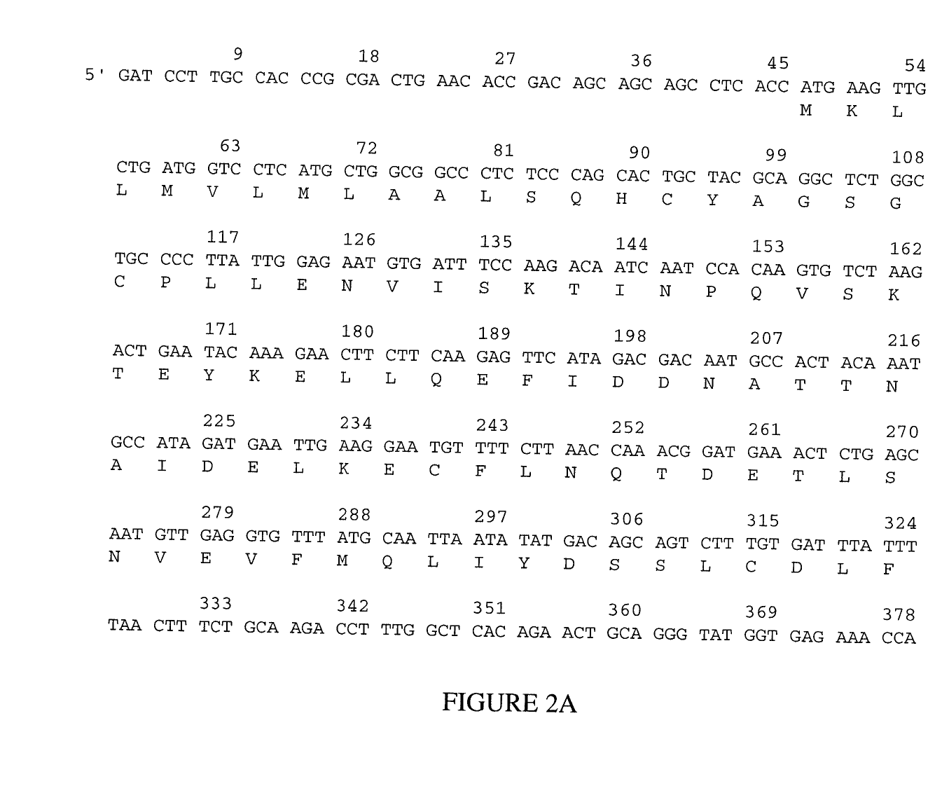

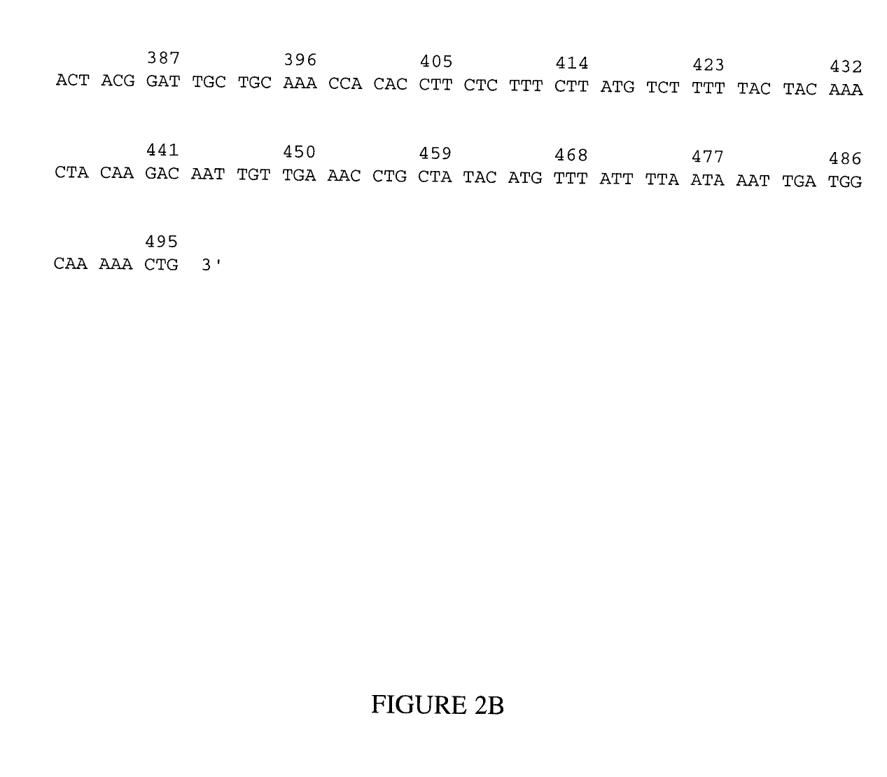

FIGS. 2A and 2B show the amino acid sequence (SEQ ID NO:3) and nucleic acid sequence (SEQ ID NO:4) of human steroid binding protein C2, hSBP2 (MacDNAsis software, Hitachi Software Engineering Co Ltd).

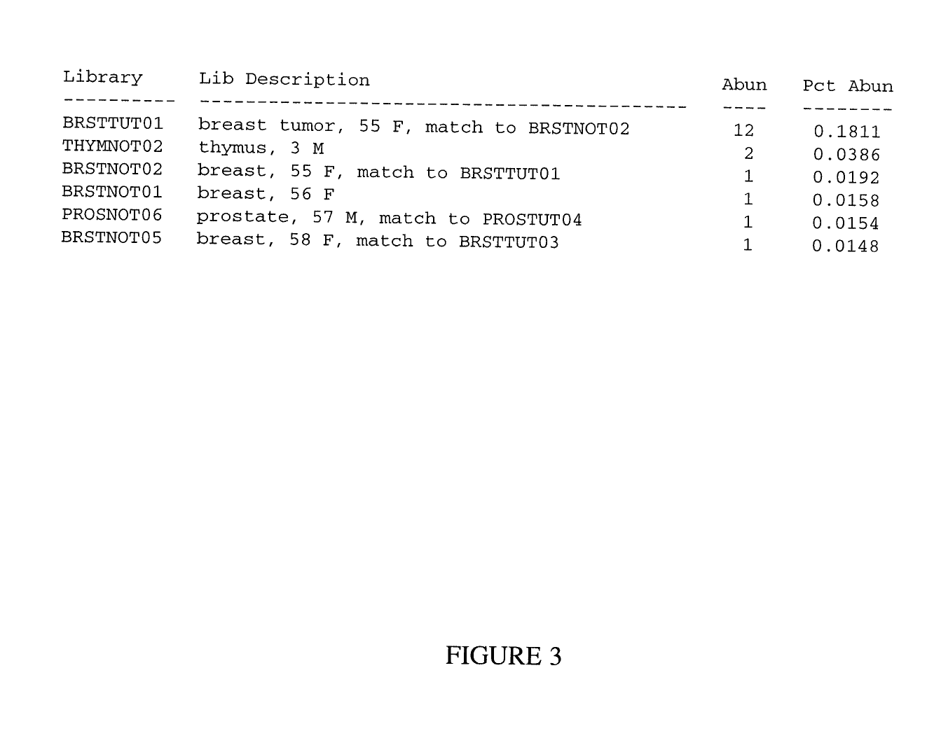

FIG. 3 shows the northern analysis for the consensus sequence (SEQ ID NO:2) for hSBP1 (Incyte clone 606491). The northern analysis was produced electronically using LIFESEQ™ database (Incyte Pharmaceuticals, Palo Alto Calif.). The abundance data (Abun) represent the number of transcripts of the gene of interest in the cDNA library. Percent abundance is calculated by dividing the number of transcripts of a gene of interest present in a cDNA library by the total number of transcripts in the cDNA library.

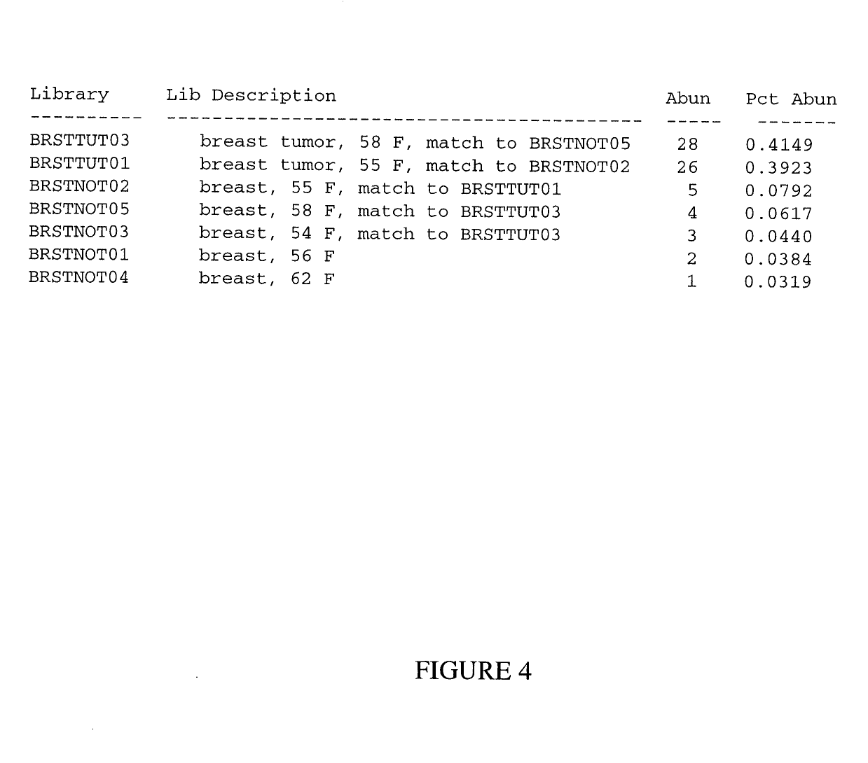

FIG. 4 shows the northern analysis for the consensus sequence (SEQ ID NO:4) (LIFESEQ™ database, Incyte Pharmaceuticals, Palo Alto Calif.).

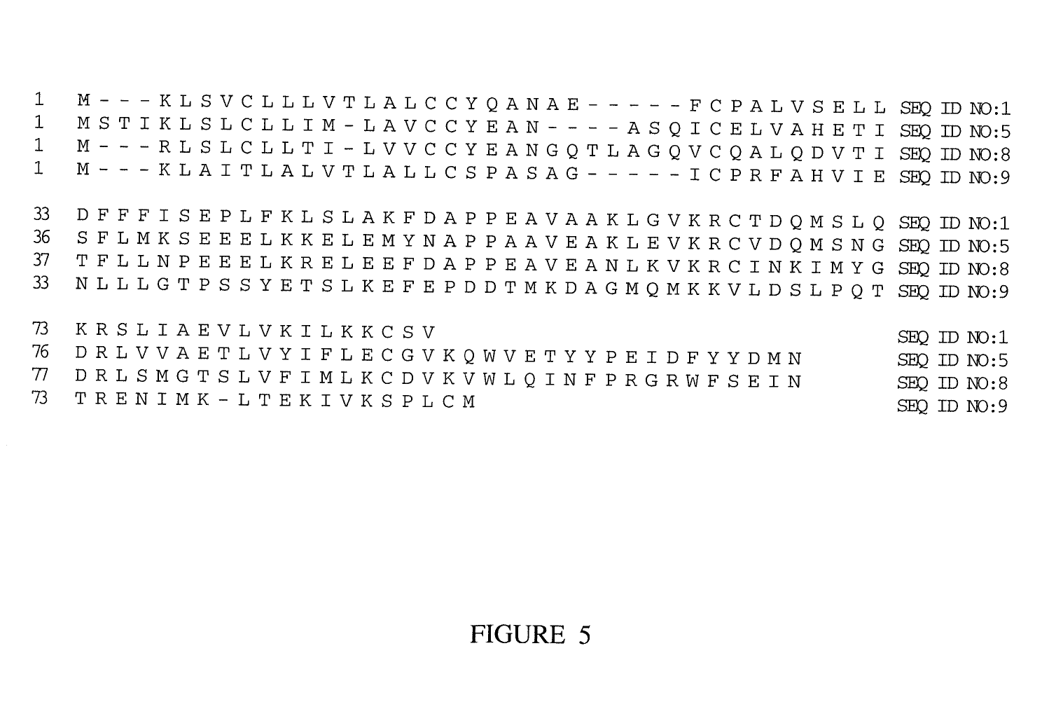

FIG. 5 shows the amino acid sequence alignments among hSBP1 (606491; SEQ ID NO:1) rat prostatic binding proteins C1 and C2 (SEQ ID NOS:5 and 8), and rabbit uteroglobin (SEQ ID NO:9), produced using the multisequence alignment program of DNAStar software (DNAStar Inc, Madison Wis.).

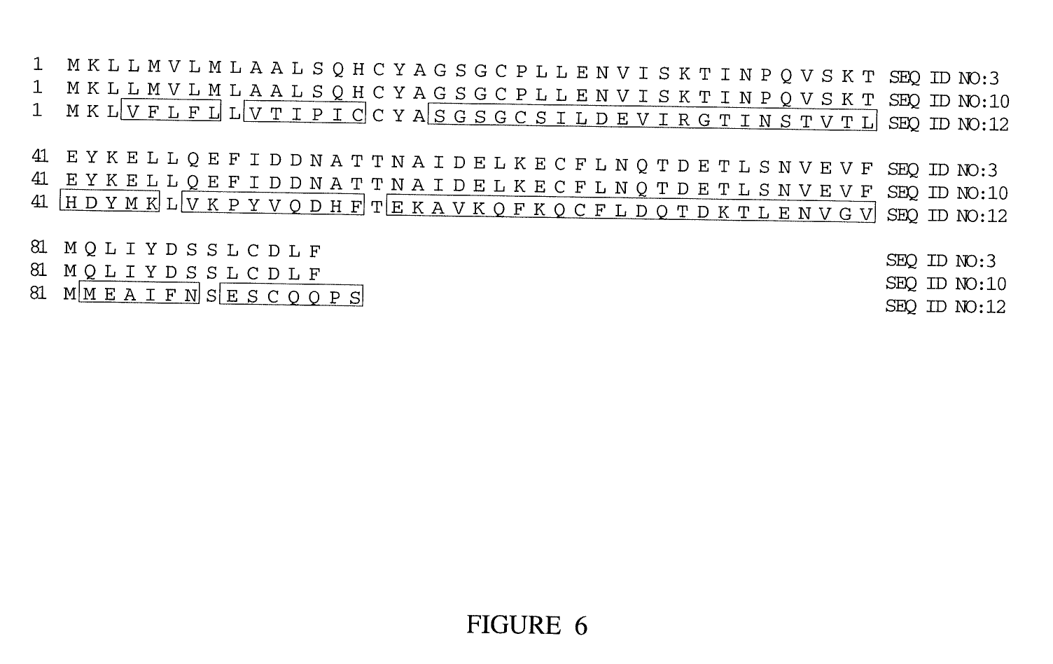

FIG. 6 shows the amino acid sequence alignments among hSBP2 (SEQ ID NO:3) human mammaglobin (GI 1199596; SEQ ID NO:10), and rat prostatic binding protein C3 (GI 206453; SEQ ID NO:12), produced using the multisequence alignment program of DNAStar software (DNAStar Inc, Madison Wis.).

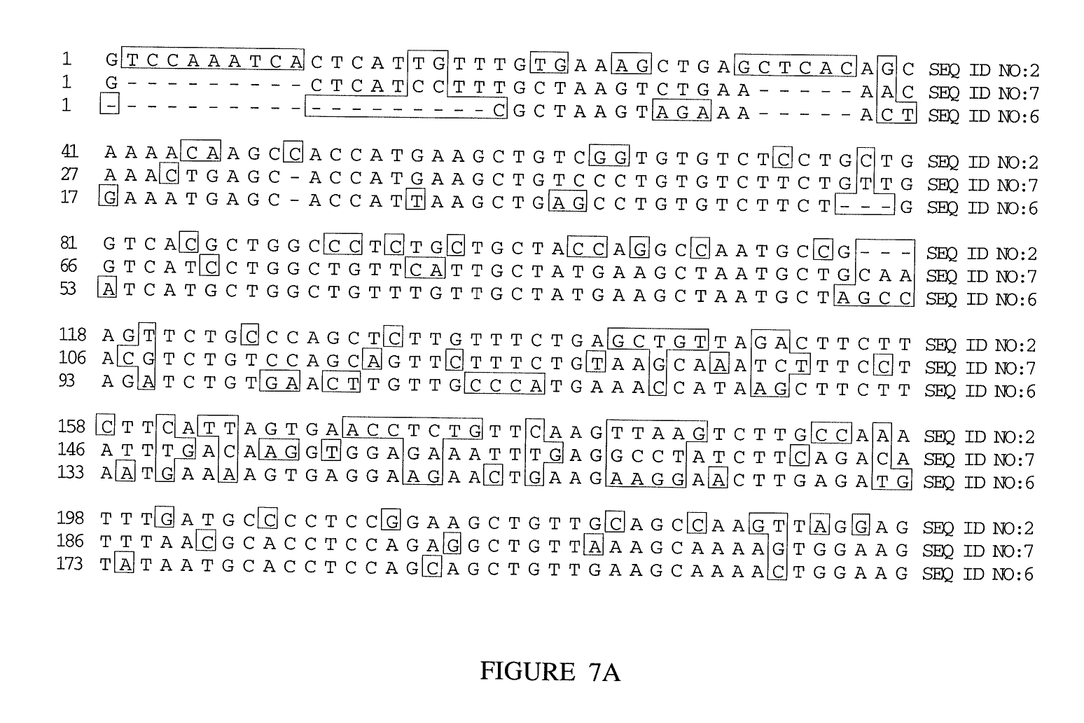

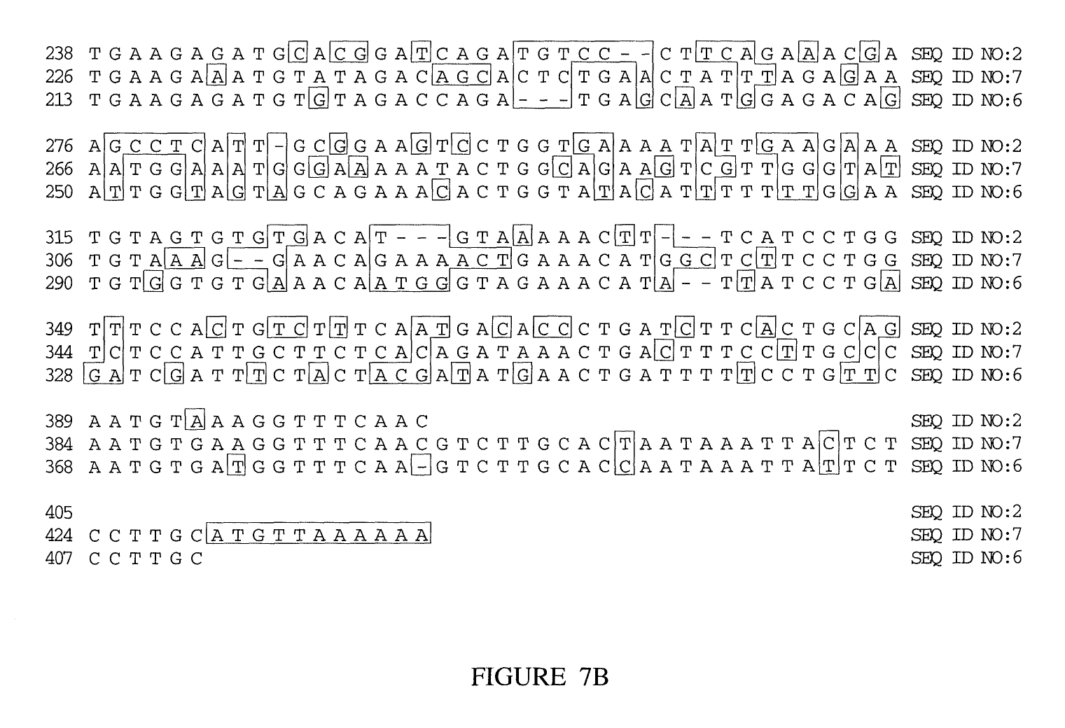

FIGS. 7A and 7B show the nucleotide sequence alignments between hSBP1 (606491; SEQ ID NO:2), hamster FHG22 (GI 1045204; SEQ ID NO:7), and rat prostatic binding protein C1 (GI 206441; SEQ ID NO:6).

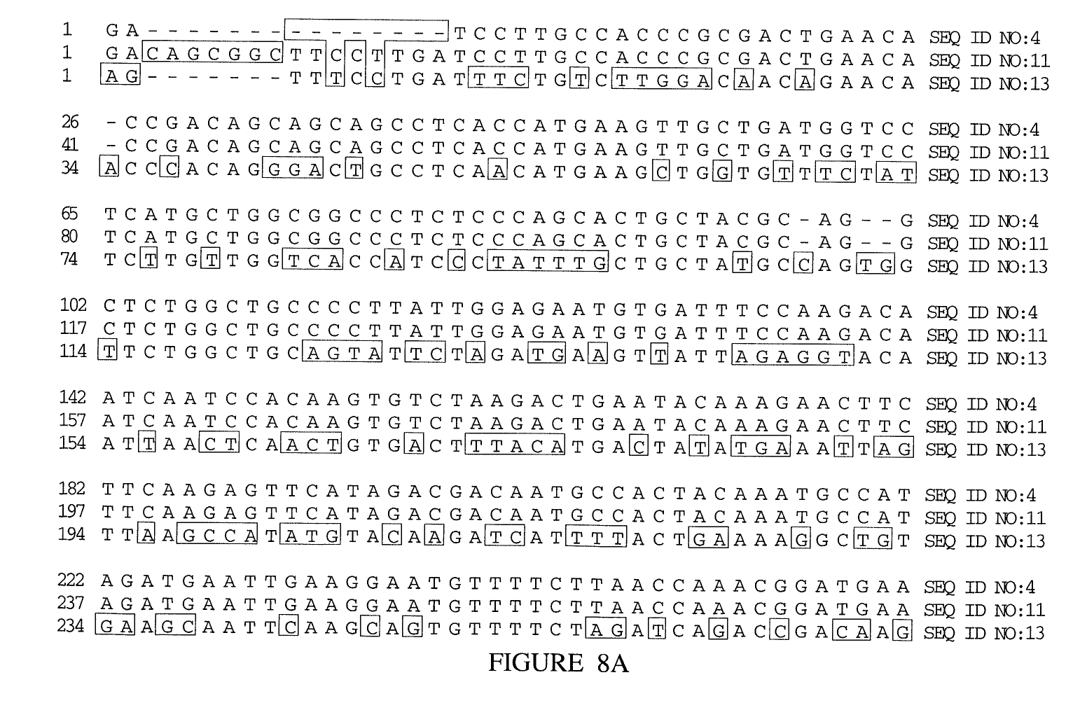

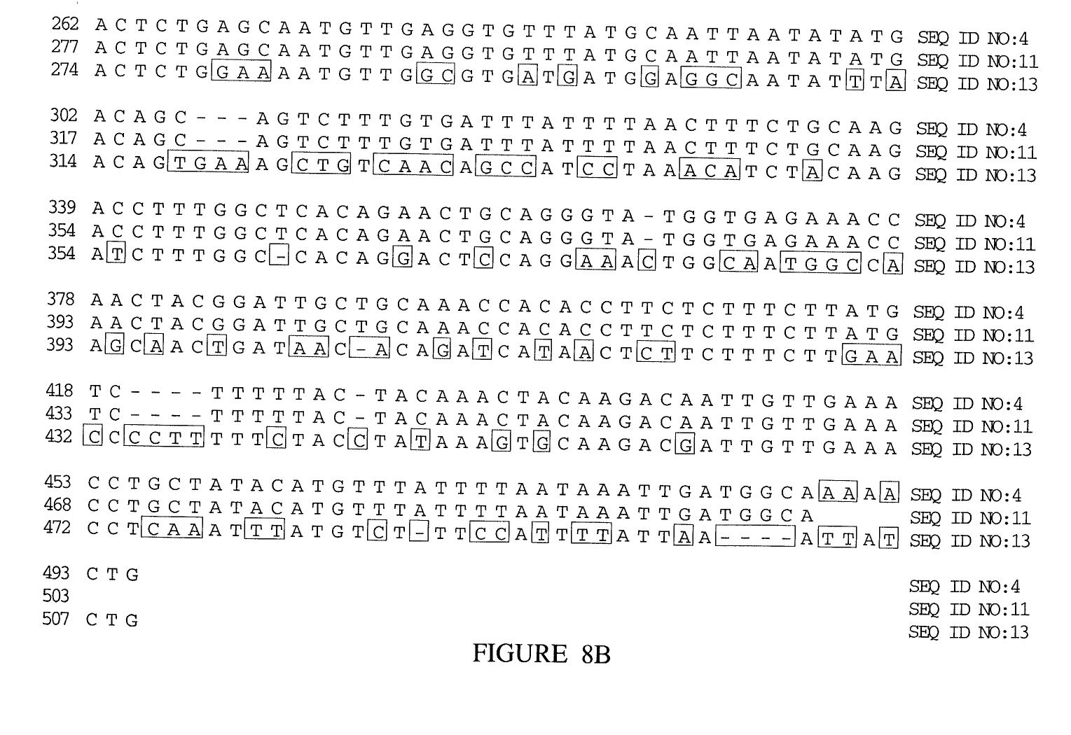

FIGS. 8A and 8B show the nucleotide sequence alignments between hSBP2 (602516; SEQ ID NO:4), human mammaglobin (GI 1199595; SEQ ID NO:11), and rat prostatic binding protein C3 (GI 206452; SEQ ID NO:13).

### DETAILED DESCRIPTION OF THE INVENTION

### Definitions

“Nucleic acid sequence” as used herein refers to an oligonucleotide, nucleotide or polynucleotide, and fragments or portions thereof, and to DNA or RNA of genomic or synthetic origin which can be single- or double-stranded, and represent the sense or antisense strand. Similarly, “amino acid sequence” as used herein refers to an oligopeptide, peptide, polypeptide, or protein sequence. Where “amino acid sequence” is recited herein to refer to an amino acid sequence of a naturally-occurring protein molecule, “amino acid sequence” and like terms (e.g., polypeptide, or protein) are not meant to limit the amino acid sequence to the complete, native amino acid sequence associated with the recited protein molecule.

“Peptide nucleic acid” as used herein refers to a molecule which comprises an oligomer to which an amino acid residue, such as lysine, and an amino group have been added. These small molecules, also designated anti-gene agents, stop transcript elongation by binding to their complementary (template) strand of nucleic acid (Nielsen P E et al (1993) Anticancer Drug Des 8:53-63).

As used herein, “SBP” refers to the amino acid sequences of substantially purified steroid binding protein obtained from any species, particularly mammalian, including bovine, ovine, porcine, murine, equine, and preferably human, from any source whether natural, synthetic, semi-synthetic or recombinant. The term “hSBP” as used herein refers to human steroid binding protein and is meant to encompass hSBP1 and hSBP2 polypeptides collectively.

As used herein, “antigenic amino acid sequence” means an amino acid sequence that, either alone or in association with a carrier molecule, can elicit an antibody response in a mammal.

A “variant” of hSBP is defined as an amino acid sequence that is altered by one or more amino acids. The variant can have “conservative” changes, wherein a substituted amino acid has similar structural or chemical properties, e.g., replacement of leucine with isoleucine. More rarely, a variant can have “nonconservative” changes, e.g., replacement of a glycine with a tryptophan. Similar minor variations can also include amino acid deletions or insertions, or both. Guidance in determining which and how many amino acid residues may be substituted, inserted or deleted without abolishing biological or immunological activity can be found using computer programs well known in the art, for example, DNAStar software.

A “deletion” is defined as a change in either amino acid or nucleotide sequence in which one or more amino acid or nucleotide residues, respectively, are absent.

An “insertion” or “addition” is that change in an amino acid or nucleotide sequence which has resulted in the addition of one or more amino acid or nucleotide residues, respectively, as compared to the naturally occurring hSBP.

A “substitution” results from the replacement of one or more amino acids or nucleotides by different amino acids or nucleotides, respectively.

The term “biologically active” refers to a hSBP having structural, regulatory, or biochemical functions of a naturally occurring hSBP. Likewise, “immunologically active” defines the capability of the natural, recombinant or synthetic hSBP, or any oligopeptide thereof, to induce a specific immune response in appropriate animals or cells and to bind with specific antibodies.

The term “derivative” as used herein refers to the chemical modification of a nucleic acid encoding hSBP or the encoded hSBP. Illustrative of such modifications would be replacement of hydrogen by an alkyl, acyl, or amino group. A nucleic acid derivative would encode a polypeptide which retains essential biological characteristics of natural hSBP.

As used herein, the term “substantially purified” refers to molecules, either nucleic or amino acid sequences, that are removed from their natural environment, isolated or separated, and are at least 60% free, preferably 75% free, and most preferably 90% free from other components with which they are naturally associated.

“Stringency” typically occurs in a range from about Tm−5° C. (5° C. below the Tm of the probe)to about 20° C. to 25° C. below Tm. As will be understood by those of skill in the art, a stringency hybridization can be used to identify or detect identical polynucleotide sequences or to identify or detect similar or related polynucleotide sequences.

The term “hybridization” as used herein shall include “any process by which a strand of nucleic acid joins with a complementary strand through base pairing” (Coombs J (1994) Dictionary of Biotechnology, Stockton Press, New York N.Y.). Amplification as carried out in the polymerase chain reaction technologies is described in Dieffenbach C W and G S Dveksler (1995, PCR Primer, a Laboratory Manual, Cold Spring Harbor Press, Plainview N.Y.).

### Preferred Embodiments

The present invention relates to hSBP and to the use of hSBP nucleic acid and amino acid sequences in the study, diagnosis, prevention and treatment of disease. cDNAs encoding a portion of hSBP were predominantly found in cDNA libraries derived from breast tumor tissue (FIGS. 3 and 4). The abundance data (Abun) reflects the relative level of expression the hSBP sequence in the breast, thymus and prostatic cDNA libraries, with the percentage abundance (Pct Abun) representing the percent of total expressed mRNAs that are homologous to the hSBP sequence.

The present invention also encompasses hSBP variants. A preferred hSBP variant is one having at least 80% amino acid sequence similarity to an amino acid sequence of an hSBP (i.e., an hSBP1 amino acid sequence (SEQ ID NO:1) or an hSBP2 amino acid sequence (SEQ ID NO:3). A more preferred hSBP variant is one having at least 90% amino acid sequence similarity to SEQ ID NO:1 or SEQ ID NO:3. A most preferred hSBP variant is one having at least 95% amino acid sequence similarity to SEQ ID NO:1 or SEQ ID NO:3.

Nucleic acids encoding the human hSBP of the present invention were first identified in cDNA, Incyte Clones 606491 and 602615 from breast tumor cell cDNA library BRSTTUTO1 through a computer-generated search for amino acid sequence alignments. A consensus sequence for each of hSBP1 (SEQ ID NO:2) and hSBP2 (SEQ ID NO:4) was derived from the overlapping and/or extended nucleic acid sequences as shown in the tables below.

TABLE 1
Clones from which the consensus sequence (SEQ ID NO:2) of hSBP-C1 was derived.

| Sequence I.D. | cDNA Library | Sequence I.D. | cDNA Library | Sequence I.D. | cDNA Library |
|---|---|---|---|---|---|
| 419412H1 | BRSTNOT01 | 606371H1 | BRSTTUT01 | 1212741H1 | BRSTTUT01 |
| 603148H1 | BRSTTUT01 | 606491H1 | BRSTTUT01 | 1215122H1 | BRSTTUT01 |
| 603224H1 | BRSTTUT01 | 825519H1 | PROSNOT06 | 1216374H1 | BRSTTUT01 |
| 604290H1 | BRSTTUT01 | 967077H1 | BRSTNOT05 | 1217152H1 | BRSTTUT01 |
| 604954H1 | BRSTTUT01 | 1209955H1 | BRSTNOT02 | | |
| 605120H1 | BRSTTUT01 | 1212005H1 | BRSTTUT01 | | |

TABLE 2
Clones from which the consensus sequence (SEQ ID NO:4) of hSBP-C2 was derived.

| Sequence I.D. | cDNA Library | Sequence I.D. | cDNA Library | Sequence I.D. | cDNA Library |
|---|---|---|---|---|---|
| 410758H1 | BRSTNOT01 | 899784 | BRSTTUT03 | 968163H1 | BRSTNOT05 |
| 419059H1 | BRSTNOT01 | | | 977969H1 | BRSTNOT02 |
| 598065H1 | BRSTNOT02 | 899895H1 | BRSTTUT03 | 10000571H1 | BRSTNOT03 |
| 601000H1 | BRSTNOT02 | 900118H1 | BRSTTUT03 | 1002776H1 | BRSTNOT03 |
| 602615H1 | BRSTTUT01 | 901009H1 | BRSTTUT03 | 1004904H1 | BRSTNOT02 |
| 603548H1 | BRSTTUT01 | 902666H1 | BRSTTUT03 | 1210748H1 | BRSTNOT02 |
| 603234H1 | BRSTTUT01 | 902354H1 | BRSTTUT03 | | |
| 603999H1 | BRSTTUT01 | 959213H1 | BRSTTUT03 | 1212473H1 | BRSTTUT01 |
| 605093H1 | BRSTTUT01 | 959506H1 | BRSTTUT03 | 1213350H1 | BRSTTUT01 |
| 605204H1 | BRSTTUT01 | 960045H1 | BRSTTUT03 | 1213570H1 | BRSTTUT01 |
| 605215H1 | BRSTTUT01 | 960118H1 | BRSTTUT03 | 1213702H1 | BRSTTUT01 |
| 605561H1 | BRSTTUT01 | 960656H1 | BRSTTUT03 | 1214253H1 | BRSTTUT01 |
| 606191H1 | BRSTTUT01 | 962153H1 | BRSTTUT03 | 1214304H1 | BRSTTUT01 |
| 606289H1 | BRSTTUT01 | 962283H1 | BRSTTUT03 | 1214401H1 | BRSTTUT01 |
| 606611H1 | BRSTTUT01 | 962488H1 | BRSTTUT03 | 1215366H1 | BRSTTUT01 |
| 606664H1 | BRSTTUT01 | 962656H1 | BRSTTUT03 | 1215626H1 | BRSTTUT01 |
| 607089H1 | BRSTTUT01 | 962907H1 | BRSTTUT03 | 1216546H1 | BRSTTUT01 |
| 897552H1 | BRSTNOT05 | 963043H1 | BRSTTUT03 | 1216653H1 | BRSTTUT01 |
| 898516H1 | BRSTTUT03 | 963046H1 | BRSTTUT03 | 1216659H1 | BRSTTUT01 |
| 898821H1 | BRSTTUT03 | 964108H1 | BRSTTUT03 | 1216778H1 | BRSTTUT01 |
| 899628H1 | BRSTTUT03 | 968127H1 | BRSTNOT05 | | |

The nucleic acid sequence of SEQ ID NO:2 encodes the hSBP1 amino acid sequence, SEQ ID NO:1. The nucleic acid sequence of SEQ ID NO:4 encodes the hSBP2 amino acid sequence, SEQ ID NO:3.

The present invention is based, in part, on the chemical and structural homology between: 1) The amino acid sequence of hSBP1 and rat prostatic binding protein C1 (GI 206442; Delaey et al. 1983 Eur J Biochem 133:645-649) rat prostatic binding protein C2 (Delaey et al. 1987 Nucl Acid Res 15:1627-1641 and rabbit uteroglobin (Menne et al. 1982 Proc Natl Acad Sci USA 79:4853-4857; FIG. 5) and the amino acid sequences of hSBP2, human mammaglobin (GI 1199596; SEQ ID NO:10) and rat prostatic binding protein C3 (GI 206453; SEQ ID NO:12; FIG. 6); and 2) The nucleotide sequence encoding hSBP1, rat prostatic binding protein C1 (GI 206442; Delaey et al. supra), and hamster FHG22 (GI 1045204; Dominguez 1995 FEBS Letters 376:257-263; FIGS. 7A and 7B); and hSBP2, human mammaglobin (GI 1199595; Watson et al. 1996 Cancer Res 56:860-865), and rat prostatic binding protein C3 (GI 206452; Parker et al. 1983 J Biol Chem 258:12-15) (FIGS. 8A and 8B).

Rat prostatic binding protein (rPBP) is a tetrameric, steroid-binding glycoprotein found in rat ventral prostate, and is the principal protein in rat prostatic fluid (Delaey et al. supra; Parker et al. supra; Heyns et al. 1977 Eur J Biochem 78:221-230; Heyns et al. 1977 Biochem Biophys Res Commun 77:1492-1499; Parker et al. 1978 Eur J Biochem 85:399-406). The rPBP tetramer is composed of two subunits: one subunit containing the polypeptides C1 and C3; and the other subunit containing the polypeptides C2 and C3 (Heyns et al. 1978 Eur J Biochem 89:181-186). rPBP C3 is homologous to human mammaglobin, which in turn is homologous to human Clara cell 10-kilodalton protein and rabbit uteroglobin (Watson et al. supra).

Although rat PBP is primarily expressed in the testes (Lindzey et al. 1994 Vitamins Hormones 49:383-32), transgenic animals harboring a construct containing the 5′ flanking region of the rat PBP-C3 gene linked to the coding region for the simian virus 40 large tumor antigen express the transgene in both the prostate and the mammary gland (Allison et al. 1989 Mol Cell Biol 9(5): 2254-2257). The expression of the C3 transgene varies with the sex of the transgenic animal; male transgenic animals express the rat PBP C3 transgene in the prostate and develop prostate carcinoma, while the females express the transgene in the mammary gland and develop atypical mammary hyperplasia (Maroulakou et al. 1994 Proc Natl Acad Sci USA 91:11236-40). Expression of rPBP is regulated by androgenic steroid (e.g., testosterone) partly by stimulating rates of transcription and partly by effects on RNA stability (Parker et al. 1977 Cell 12:401-407; Heyns et al. 1977 Biochem Biophys Res Commun 77:1492-1499; Parker et al. 1979 Proc Natl Acad Sci USA 76:1580-1584; Page et al. 1982 Mol Cell Endocr 27:343-355).

rPBP is similar to estramucine binding protein (EMBP) (Heyns et al. 1977 Eur J Biochem 78:221-30). EMBP is a 46-kDa heterodimer consisting of two closely related subunits, which upon reductive cleavage of disulfide bridges, each subunit is divided into two components. The subunits differ with respect to the components C1 and C2, but share C3 (Bjork et al. 1995 The Prostate (1995) 27:70-83). EMBP binds estramucine (Appelgren et al. 1979 Acta Pharmacol Toxicol 43:368-74; Försgren et al. 1979 Cancer Res 39:5155-64; Høisaeter et al. 1981 J Steroid Biochem 14: 251-60), but does not bind free estrogens (Høisaeter et al. 1981 J Steroid Biochem 14:251-260; Försgren et al. 1979 Proc Natl Acad Sci USA 76:3149-3150). Estramucine, a nitrogen mustard derivative of 17β-estradiol (Mittelman et al. 1977 Cancer Treat Rep 61:307-10; Johnson et al. 1971 Scand J Urol Nephrol 5:103-7), is used to treat patients with prostatic carcinoma. Expression of EMBP is androgen-regulated; this androgen-dependency of EMBP tends to decline with the transformation of prostatic tissue into biologically more malignant disease (Shiina et al. 1996 Brit J Urol 77:96-101). The ratio of EMBP to dihydroxytestosterone is an indicator of the malignant potential of prostatic carcinoma (Shiina et al. supra).

Rabbit uteroglobin, a homodimeric protein coupled by two disulfide linkages, binds progesterone and structurally related steroids, is also a substrate for transglutaminases, inhibits phospholipase A₂ activity, and may interfere with the immune and inflammatory activity of several cell types (Miele et al. 1994 J Endocrinol Invest 17:679-692; Miele et al. 1987 Endocrinol Rev 8:474-490). Expression of uteroglobin is regulated by tissue-specific response to steroid hormones (Sandmoller et al. 1994 Oncogene 9:2805-2815).

FHG22 protein was isolated from a female minus male subtracted cDNA library obtained from the sexually dimorphic Syrian hamster Harderian glands (Dominguez supra). FHG nucleotide and amino acid sequence are similar to the subunits from rat prostatic steroid binding protein C1, uteroglobin (Miele et al. 1994 J Endocrinol Invest 17:679-692), major cat allergen Fel dI (chain I), and mouse salivary androgen binding proteins (subunit α) (Karn et al. 1993 Biochem Genet 32:271-277; Dominguez supra). Expression of FHG22 is tissue and sex-dependent (Dominguez supra). hSBP1 and rat prostatic binding protein C1 share 55% nucleotide sequence identity at the nucleotide sequence level, whereas hSBP1 and hamster FHG22 share 72% nucleotide sequence identity. hSBP1 is 90 amino acids in length; the amino acid sequence of hSBP1 has 49% identity with the amino acid sequence of rat prostatic binding protein C1 (SEQ ID NO:5), 44% identity with the amino acid sequence of rat prostatic binding protein C2 (SEQ ID NO:8), and 28% identity with the amino acid sequence of rabbit uteroglobin (SEQ ID NO:9) (FIG. 5).

hSBP2 is 93 amino acids in length and shares 99% nucleotide sequence identity with human mammaglobin; the nucleotide sequence of hSBP2 is about 43% identical to the nucleotide sequence of rat prostatic binding protein C3 (FIGS. 8A and 8B). The amino acid sequence of hSBP2 is 62% identical to the amino acid sequence of rat prostatic protein C3, and 100% identical to the amino acid sequence of human mammaglobin (FIG. 6). Thus, hSBP-C3 is identical to human mammaglobin.

### The hSBP Coding Sequences

The nucleic acid and deduced amino acid sequences of hSBP are shown in FIG. 1 (hSBP1) and FIGS. 2A and 2B (hSBP2). In accordance with the invention, any nucleic acid sequence that encodes an amino acid sequence of an hSBP polypeptide can be used to generate recombinant molecules which express an hSBP polypeptide. In specific embodiments described herein, a nucleotide sequence encoding a portion of hSBP1 was first isolated as Incyte Clone 606491 from a breast tumor cell line cDNA library BRSTTUT01; and a nucleotide sequence encoding a portion of hSBP2 was first isolated as Incyte Clone 602615 from a breast tumor cell line cDNA library BRSTTUT10.

It will be appreciated by those skilled in the art that as a result of the degeneracy of the genetic code, a multitude of degenerate variants of hSBP-encoding nucleotide sequences, some bearing minimal homology to the nucleotide sequences of any known and naturally occurring gene, can be produced. The invention contemplates each and every possible variation of nucleotide sequence that can be made by selecting combinations based on possible codon choices. These combinations are made in accordance with the standard triplet genetic code as applied to the nucleotide sequence of naturally occurring hSBP, and all such variations are to be considered as being specifically disclosed herein.

Although nucleotide sequences that encode hSBP and its variants are preferably capable of hybridizing to the nucleotide sequence of the naturally occurring hSBP under appropriately selected conditions of stringency, it may be advantageous to produce nucleotide sequences encoding hSBP or its derivatives possessing a substantially different codon usage. Codons can be selected to increase the rate at which expression of the peptide occurs in a particular prokaryotic or eukaryotic expression host in accordance with the frequency with which particular codons are utilized by the host. Other reasons for substantially altering the nucleotide sequence encoding hSBP and its derivatives without altering the encoded amino acid sequences include the production of RNA transcripts having more desirable properties (e.g., increased half-life) than transcripts produced from the naturally occurring sequence.

It is now possible to produce a nucleotide sequence encoding an hSBP polypeptide and/or its derivatives entirely by synthetic chemistry, after which the synthetic gene can be inserted into any of the many available DNA vectors and expression systems using reagents that are well known in the art at the time of the filing of this application. Moreover, synthetic chemistry can be used to introduce mutations into a sequence encoding an hSBP polypeptide.

Also included within the scope of the present invention are polynucleotide sequences that are capable of hybridizing to the nucleotide sequences of FIG. 1 and/or FIGS. 2A and 2B under various conditions of stringency. Hybridization conditions are based on the melting temperature (Tm) of the nucleic acid binding complex or probe, as taught in Berger and Kimmel (1987, Guide to Molecular Cloning Techniques, Methods in Enzymology, Vol 152, Academic Press, San Diego Calif.) incorporated herein by reference, and can be used at a defined stringency.

Altered nucleic acid sequences encoding hSBP that can be used in accordance with the invention include deletions, insertions or substitutions of different nucleotides resulting in a polynucleotide that encodes the same or a functionally equivalent hSBP. The protein can also comprise deletions, insertions or substitutions of amino acid residues that result in a polypeptide that is functionally equivalent to hSBP. Deliberate amino acid substitutions can be made on the basis of similarity in polarity, charge, solubility, hydrophobicity, hydrophilicity, and/or the amphipathic nature of the residues with the proviso that biological activity of hSBP is retained. For example, negatively charged amino acids include aspartic acid and glutamic acid; positively charged amino acids include lysine and arginine; and amino acids with uncharged polar head groups having similar hydrophilicity values include leucine, isoleucine, valine; glycine, alanine; asparagine, glutamine; serine, threonine phenylalanine, and tyrosine.

Alleles of hSBP are also encompassed by the present invention. As used herein, an “allele” or “allelic sequence” is an alternative form of hSBP. Alleles result from a mutation (i.e., an alteration in the nucleic acid sequence) and generally produce altered mRNAs and/or polypeptides that may or may not have an altered structure or function relative to naturally-occurring hSBP. Any given gene may have none, one, or many allelic forms. Common mutational changes that give rise to alleles are generally ascribed to natural deletions, additions or substitutions of amino acids. Each of these types of changes may occur alone or in combination with the other changes, and may occur once or multiple times in a given sequence.

Methods for DNA sequencing are well known in the art and employ such enzymes as the Klenow fragment of DNA polymerase I, Sequenase® (US Biochemical Corp, Cleveland Ohio), Taq polymerase (Perkin Elmer, Norwalk Conn.), thermostable T7 polymerase (Amersham, Chicago Ill.), or combinations of recombinant polymerases and proofreading exonucleases such as the ELONGASE Amplification System marketed by Gibco BRL (Gaithersburg Md.). Preferably, the process is automated with machines such as the Hamilton Micro Lab 2200 (Hamilton, Reno Nev.), Peltier Thermal Cycler (PTC200; MJ Research, Watertown Mass.) and the ABI 377 DNA sequencers (Perkin Elmer).

### Extending the Polynucleotide Sequence

The polynucleotide sequence encoding hSBP can be extended utilizing partial nucleotide sequence and various methods known in the art to detect upstream sequences such as promoters and regulatory elements. Clones that contain extended sequences are designated by a suffix (see the tables above). Gobinda et al (1993; PCR Methods Applic 2:318-22) disclose “restriction-site” polymerase chain reaction (PCR) as a direct method which uses universal primers to retrieve unknown sequence adjacent to a known locus. First, genomic DNA is amplified in the presence of primer to a linker sequence and a primer specific to the known region. The amplified sequences are subjected to a second round of PCR with the same linker primer and another specific primer internal to the first one. Products of each round of PCR are transcribed with an appropriate RNA polymerase and sequenced using reverse transcriptase.

Inverse PCR can be used to amplify or extend sequences using divergent primers based on a known region (Triglia T et al (1988) Nucleic Acids Res 16:8186). The primers can be designed using OLIGO® 4.06 Primer Analysis Software (1992; National Biosciences Inc, Plymouth Minn.), or another appropriate program, to be 22-30 nucleotides in length, to have a GC content of 50% or more, and to anneal to the target sequence at temperatures about 68°-72° C. This method uses several restriction enzymes to generate a suitable fragment in the known region of a gene. The fragment is then circularized by intramolecular ligation and used as a PCR template.

Capture PCR (Lagerstrom M et al (1991) PCR Methods Applic 1:111-19) is a method for PCR amplification of DNA fragments adjacent to a known sequence in human and yeast artificial chromosome DNA. Capture PCR also requires multiple restriction enzyme digestions and ligations to place an engineered double-stranded sequence into an unknown portion of the DNA molecule before PCR.

Another method that can be used to retrieve unknown sequences is that of Parker J D et al (1991; Nucleic Acids Res 19:3055-60). Additionally, one can use PCR, nested primers, and PromoterFinder libraries to “walk in” genomic DNA (PromoterFinder™ Clontech (Palo Alto Calif.). This process avoids the need to screen libraries and is useful in finding intron/exon junctions. Preferably, the libraries used to identify full length cDNAs have been size-selected to include larger cDNAs. More preferably, the cDNA libraries used to identify full-length cDNAs are those generated using random primers, in that such libraries will contain more sequences comprising regions 5′ of the sequence(s) of interest. A randomly primed library can be particularly useful where oligo d(T) libraries do not yield a full-length cDNA. Genomic libraries are preferred for identification and isolation of 5′ nontranslated regulatory regions of a sequence(s) of interest.

Capillary electrophoresis can be used to analyze the size of, or confirm the nucleotide sequence of, sequencing or PCR products. Systems for rapid sequencing are available from Perkin Elmer, Beckman Instruments (Fullerton Calif.), and other companies. Capillary sequencing can employ flowable polymers for electrophoretic separation, four different, laser-activatable fluorescent dyes (one for each nucleotide), and a charge coupled device camera for detection of the wavelengths emitted by the fluorescent dyes. Output/light intensity is converted to electrical signal using appropriate software (e.g. Genotyper™ and Sequence Navigator™ from Perkin Elmer). The entire process from loading of the samples to computer analysis and electronic data display is computer controlled. Capillary electrophoresis is particularly suited to the sequencing of small pieces of DNA that might be present in limited amounts in a particular sample. Capillary electrophoresis provides reproducible sequencing of up to 350 bp of M13 phage DNA in 30 min (Ruiz-Martinez M C et al (1993) Anal Chem 65:2851-2858).

### Expression of the Nucleotide Sequence

In accordance with the present invention, polynucleotide sequences that encode hSBP polypeptides (which polypeptides include fragments of the naturally-occurring polypeptide, fusion proteins, and functional equivalents thereof) can be used in recombinant DNA molecules that direct the expression of hSBP in appropriate host cells. Due to the inherent degeneracy of the genetic code, other DNA sequences that encode substantially the same or a functionally equivalent amino acid sequence, can be used to clone and express hSBP. As will be understood by those of skill in the art, it may be advantageous to produce hSBP-encoding nucleotide sequences possessing non-naturally occurring codons. Codons preferred by a particular prokaryotic or eukaryotic host (Murray E et al (1989) Nuc Acids Res 17:477-508) can be selected, for example, to increase the rate of hSBP expression or to produce recombinant RNA transcripts having a desirable characteristic(s) (e.g., longer half-life than transcripts produced from naturally occurring sequence).

The nucleotide sequences of the present invention can be engineered in order to alter an hSBP coding sequence for a variety of reasons, including but not limited to, alterations that facilitate the cloning, processing and/or expression of the gene product. For example, mutations can be introduced using techniques that are well known in the art, e.g., site-directed mutagenesis to insert new restriction sites, alter glycosylation patterns, change codon preference, produce splice variants, etc.

In another embodiment of the invention, a natural, modified, or recombinant polynucleotide encoding an hSBP polypeptide can be ligated to a heterologous sequence to encode a fusion protein. For example, where an hSBP polypeptide is to be used in a peptide library for screening and identification of inhibitors of hSBP activity, it may be desirable to provide the hSBP polypeptide in the peptide library as a chimeric hSBP protein that can be recognized by a commercially available antibody. A fusion protein can also be engineered to contain a cleavage site located between an hSBP polypeptide-encoding sequence and a heterologous polypeptide sequence, such that the hSBP polypeptide can be cleaved and purified away from the heterologous moiety.

In an alternative embodiment of the invention, a nucleotide sequence encoding an hSBP polypeptide can be synthesized, in whole or in part, using chemical methods well known in the art (see Caruthers et al (1980) Nuc Acids Res Symp Ser 215-23, Horn et al(1980) Nuc Acids Res Symp Ser 225-32, etc.). Alternatively, the polypeptide itself can be produced using chemical methods to synthesize an hSBP amino acid sequence, in whole or in part. For example, peptide synthesis can be performed using various solid-phase techniques (Roberge et al (1995) Science 269:202-204) and automated synthesis can be achieved, for example, using the ABI 431A Peptide Synthesizer (Perkin Elmer) in accordance with the instructions provided by the manufacturer.

The newly synthesized peptide can be substantially by preparative high performance liquid chromatography (e.g., Creighton (1983) Proteins, Structures and Molecular Principles, WH Freeman and Co, New York N.Y.). The composition of the synthetic peptides can be confirmed by amino acid analysis or sequencing (e.g., the Edman degradation procedure; Creighton, supra). Additionally the amino acid sequence of hSBP, or any part thereof, can be altered during direct synthesis and/or combined using chemical methods with sequences from other proteins, or any part thereof, to produce a variant polypeptide.

### Expression Systems

In order to express a biologically active hSBP polypeptide, the nucleotide sequence encoding an hSBP polypeptide or its functional equivalent, is inserted into an appropriate expression vector, i.e., a vector having the necessary elements for the transcription and translation of the inserted coding sequence.

Methods well known to those skilled in the art can be used to construct expression vectors comprising an hSBP polypeptide-encoding sequence and appropriate transcriptional or translational controls. These methods include in vitro recombinant DNA techniques, synthetic techniques and in vivo recombination or genetic recombination. Such techniques are described in Sambrook et al (1989) Molecular Cloning, A Laboratory Manual, Cold Spring Harbor Press, Plainview N.Y. and Ausubel F M et al (1989) Current Protocols in Molecular Biology, John Wiley & Sons, New York N.Y.

A variety of expression vector/host systems can be utilized to express an hSBP polypeptide-encoding sequence. These include, but are not limited to, microorganisms such as bacteria transformed with recombinant bacteriophage, plasmid or cosmid DNA expression vectors; yeast transformed with yeast expression vectors; insect cell systems infected with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti or pBR322 plasmid); or animal cell systems.

The “control elements” or “regulatory sequences” of these systems, which vary in their strength and specificities, are those nontranslated regions of the vector, enhancers, promoters, and 3′ untranslated regions that interact with host cellular proteins to facilitate transcription and translation of a nucleotide sequence of interest. Depending on the vector system and host utilized, any number of suitable transcriptional and translational elements, including constitutive and inducible promoters, can be used. For example, when cloning in bacterial systems, inducible promoters such as the hybrid lacZ promoter of the Bluescript® phagemid (Stratagene, La Jolla Calif.) or pSport1 (Gibco BRL), ptrp-lac hybrids, and the like can be used. The baculovirus polyhedron promoter can be used in insect cells. Promoters or enhancers derived from the genomes of plant cells (e.g., heat shock, RUBISCO; and storage protein genes) or from plant viruses (e.g., viral promoters or leader sequences) can be cloned into the vector. In mammalian cell systems, promoters from the mammalian genes or from mammalian viruses are most appropriate. Where it is desirable to generate a cell line containing multiple copies of an hSBP polypeptide-encoding sequence, vectors derived from SV40 or EBV can be used in conjunction with other optional vector elements, e.g., an appropriate selectable marker.

In bacterial systems, a number of expression vectors can be used to express an hSBP polypeptide of interest, and will vary with a variety of factors including the intended use intended for the hSBP polypeptide produced. For example, when large quantities of an hSBP polypeptide are required (e.g., for the antibody production), vectors that direct high-level expression of fusion proteins that can be readily purified may be desirable. Such vectors include, but are not limited to, the multifunctional E. coli cloning and expression vectors such as Bluescript® (Stratagene; which provides for in-frame ligation of a hSBP polypeptide-encoding sequence with sequences encoding the amino-terminal Met and the subsequent 7 residues of β-galactosidase, thereby producing an hSBP polypeptide-β-galactosidase hybrid protein); pIN vectors (Van Heeke & Schuster (1989) J Biol Chem 264:5503-5509); and the like. pGEX vectors (Promega, Madison Wis.) can also be used to express foreign polypeptides as glutathione S-transferase (GST) fusion proteins. In general, such GST fusion proteins are soluble and can be easily purified from cell lysates by adsorption to glutathione-agarose beads followed by elution in the presence of free glutathione. GST fusion proteins can be designed to include heparin, thrombin or factor XA protease cleavage sites so that the cloned polypeptide of interest can be readily separated from the GST moiety.

Where the host cell is yeast (e.g., Saccharomyces cerevisiae) a number of vectors containing constitutive or inducible promoters such as alpha factor, alcohol oxidase and PGH can be used. For reviews, see Ausubel et al (supra) and Grant et al (1987) Methods in Enzymology 153:516-544.

Where plant expression vectors are used, the expression of an hSBP polypeptide-encoding sequence can be driven by any of a number of promoters. For example, viral promoters such as the 35S and 19S promoters of CaMV (Brisson et al (1984) Nature 310:511-514) can be used alone or in combination with the omega leader sequence from TMV (Takamatsu et al (1987) EMBO J 6:307-311). Alternatively, plant promoters, such as the small subunit of RUBISCO (Coruzzi et al (1984) EMBO J 3:1671-1680; Broglie et al (1984) Science 224:838-843) or heat shock promoters (Winter J and Sinibaldi R M (1991) Results Probl Cell Differ 17:85-105), can be used. These constructs can be introduced into plant cells by direct DNA transformation or pathogen-mediated transfection. For reviews of such techniques, see Hobbs S or Murry L E in McGraw Hill Yearbook of Science and Technology (1992) McGraw Hill New York N.Y., pp 191-196 or Weissbach and Weissbach (1988) Methods for Plant Molecular Biology, Academic Press, New York N.Y., pp 421-463.

Alternatively, insect cell expression systems can be used to express an hSBP polypeptide. In one such system, Autogrgaha californica nuclear polyhedrosis virus (AcNPV) is used as a vector to express foreign genes in Spodoptera frugiperda cells or in Trichoplusia larvae. The hSBP polypeptide-encoding sequence can be cloned into a nonessential region of the virus, such as the polyhedron gene, and placed under control of the polyhedron promoter. Successful insertion of hSBP renders the polyhedron gene inactive and produces recombinant virus lacking coat protein. The recombinant viruses are then used to infect S. frugiperda cells or Trichoplusia larvae for expression of hSBP polypeptide (Smith et al (1983) J Virol 46:584; Engelhard E K et al (1994) Proc Nat Acad Sci 91:3224-7).

Where the host cell is a mammalian cells, a number of viral-based expression systems can be used. For example, the expression vector can be derived from an adenovirus nucleotide sequence. An hSBP polypeptide-encoding sequence can be ligated into an adenovirus transcription/translation complex, which is composed of the late promoter and tripartite leader sequence. Insertion of the nucleotide sequence of interest into a nonessential E1 or E3 region of the viral genome will result in the production of a viable virus capable of expressing hSBP polypeptide in infected host cells (Logan and Shenk (1984) Proc Natl Acad Sci 81:3655-59). In addition, transcriptional enhancers, such as the Rous sarcoma virus (RSV) enhancer, can be used to increase expression in mammalian host cells.

Specific initiation signals may also be required for efficient translation of an hSBP polypeptide-encoding sequence, e.g., the ATG initiation codon and flanking sequences. Where a native hSBP polypeptide encoding sequence, its initiation codon and upstream sequences are inserted into the appropriate expression vector, no additional translational control signals may be needed. However, where only coding sequence, or a portion thereof, is inserted in an expression vector, exogenous transcriptional control signals including the ATG initiation codon must be provided. Furthermore, the initiation codon must be in the correct reading frame to ensure transcription of the entire insert. Exogenous transcriptional elements and initiation codons can be derived from various origins, and can be either natural or synthetic. Expression efficiency can be enhanced by including enhancers appropriate to the cell system in use (Scharf D et al (1994) Results Probl Cell Differ 20:125-62; Bittner et al (1987) Methods in Enzymol 153:516-544).

Host cells can be selected for hSBP polypeptide expression according to the ability of the cell to modulate the expression of the inserted sequences or to process the expressed protein in a desired fashion. Such modifications of the polypeptide include, but are not limited to, acetylation, carboxylation, glycosylation, phosphorylation, lipidation and acylation. Post-translational processing that involves cleavage of a “prepro” form of the protein may also be important for correct polypeptide folding, membrane insertion, and/or function. Host cells such as CHO, HeLa, MDCK, 293, W138, and others have specific cellular machinery and characteristic mechanisms for such post-translational activities and may be chosen to ensure the correct modification and processing of the introduced, foreign polypeptide.

Where long-term, high-yield recombinant polypeptide production is desired, stable expression is preferred. For example, cell lines that stably express hSBP can be transformed using expression vectors containing viral origins of replication or endogenous expression elements and a selectable marker gene. After introduction of the vector, cells can be grown for 1-2 days in an enriched media before they are exposed to selective media. The selectable marker, which confers resistance to the selective media, allows growth and recovery of cells that successfully express the introduced sequences. Resistant, stably transformed cells can be proliferated using tissue culture techniques appropriate to the host cell type.

Any number of selection systems can be used to recover transformed cell lines. These include, but are not limited to, the herpes simplex virus thymidine kinase (Wigler M et al (1977) Cell 11:223-32) and adenine phosphoribosyltransferase (Lowy I et al (1980) Cell 22:817-23) genes which can be employed in tk- or aprt- cells, respectively. Also, antimetabolite, antibiotic or herbicide resistance can be used as the basis for selection; for example, dhfr which confers resistance to methotrexate (Wigler M et al (1980) Proc Natl Acad Sci 77:3567-70); npt, which confers resistance to the aminoglycosides neomycin and G-418 (Colbere-Garapin F et al (1981) J Mol Biol 150:1-14) and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman S C and R C Mulligan (1988) Proc Natl Acad Sci 85:8047-51). Recently, the use of visible markers has gained popularity with such markers as anthocyanins, β-glucuronidase and its substrate, GUS, and luciferase and its substrate, luciferin, being widely used not only to identify transformants, but also to quantify the amount of transient or stable protein expression attributable to a specific vector system (Rhodes CA et al (1995) Methods Mol Biol 55:121-131).

### Identification of Transformants Containing the Polynucleotide Sequence

Although the presence/absence of marker gene expression suggests that the gene of interest is also present, its presence and expression should be confirmed. For example, if the hSBP polypeptide encoding sequence is inserted within a marker gene sequence, recombinant cells containing this sequence can be identified by the absence of marker gene function. Alternatively, a marker gene can be placed in tandem with a hSBP sequence under the control of a single promoter. Expression of the marker gene in response to induction or selection is indicative of expression of the tandem hSBP.

Alternatively, host cells that contain the coding sequence for hSBP polypeptides and express hSBP polypeptides can be identified by a variety of procedures known to those of skill in the art. These procedures include, but are not limited to, DNA-DNA or DNA-RNA hybridization and protein bioassay or immunoassay techniques including membrane, solution, or chip-based technologies for the detection and/or quantitation of the nucleic acid or protein.

The presence of the polynucleotide sequence encoding hSBP polypeptides can be detected by DNA-DNA or DNA-RNA hybridization or amplification using probes, portions or fragments of polynucleotides encoding hSBP. Nucleic acid amplification-based assays involve the use of oligonucleotides or oligomers based on the hSBP polypeptide-encoding sequence to detect transformants containing hSBP polypeptide-encoding DNA or RNA. As used herein “oligonucleotides” or “oligomers” refer to a nucleic acid sequence of at least about 10 nucleotides and as many as about 60 nucleotides, preferably about 15 to 30 nucleotides, and more preferably about 20-25 nucleotides which can be used as a probe or amplimer.

A variety of protocols for detecting and measuring the expression of hSBP, using either polyclonal or monoclonal antibodies specific for the protein are known in the art. Examples include enzyme-linked immunosorbent assay (ELISA), radioimmunoassay (RIA) and fluorescent activated cell sorting (FACS). A two-site, monoclonal-based immunoassay utilizing monoclonal antibodies reactive to two non-interfering epitopes on hSBP is preferred, but a competitive binding assay can be employed. These and other assays are described in, e.g., Hampton R et al (1990, Serological Methods, a Laboratory Manual, APS Press, St Paul Minn.) and Maddox D E et al (1983, J Exp Med 158:1211).

A wide variety of detectable labels and conjugation techniques are known by in the art and can be used in various nucleic acid and amino acid assays. Means for producing labeled hybridization or PCR probes for detecting sequences related to hSBP-encoding polynucleotides include oligolabeling, nick translation, end-labeling or PCR amplification using a labeled nucleotide. Alternatively, an nucleotide sequence encoding an hSBP polypeptide can be cloned into a vector for the production of an mRNA probe. Such vectors, which are known in the art and commercially available, can be used to synthesize RNA probes in vitro by addition of an appropriate RNA polymerase such as T7, T3 or SP6 and labeled nucleotides.

A number of companies, including Pharmacia Biotech (Piscataway N.J.), Promega (Madison Wis.), and US Biochemical Corp (Cleveland Ohio), supply commercial kits and protocols suitable for the methods described above. Suitable reporter molecules or labels include those radionuclides, enzymes, fluorescent, chemiluminescent, or chromogenic agents as well as substrates, cofactors, inhibitors, magnetic particles and the like, as described in U.S. Pat. Nos. 3,817,837; 3,850,752; 3,939,350; 3,996,345; 4,277,437; 4,275,149 and 4,366,241, each of which are incorporated herein by reference. Recombinant immunoglobulins can be produced as according to U.S. Pat. No. 4,816,567, incorporated herein by reference.

### Purification of hSBP

Host cells transformed with a nucleotide sequence encoding an hSBP polypeptide can be cultured under conditions suitable for the expression and recovery of the hSBP polypeptide from cell culture. The polypeptide produced by a recombinant cell may be secreted or retained intracellularly depending on the sequence and/or the vector used. As will be understood by those of skill in the art, expression vectors containing polynucleotides encoding hSBP polypeptides can be designed with signal sequences that direct secretion of hSBP through a prokaryotic or eukaryotic cell membrane.

Recombinant hSBP constructs can also include a nucleotide sequence(s) encoding one or more polypeptide domains that, when expressed in-frame with the hSBP-encoding sequence, facilitates purification of soluble proteins (Kroll D J et al (1993) DNA Cell Biol 12:441-53; c.f. discussion of vectors infra containing fusion proteins). Such purification facilitating domains include, but are not limited to, metal chelating peptides (e.g., histidine-tryptophan modules) that allow purification with immobilized metals, protein A domains that allow purification with immobilized immunoglobulin, and the domain utilized in the FLAGS extension/affinity purification system (Immunex Corp, Seattle Wash.). A cleavable linker sequences(s) (e.g., Factor XA or enterokinase (Invitrogen, San Diego Calif.)) between the purification domain and the hSBP polypeptide-encoding sequence can be included to facilitate purification. One such expression vector provides for expression of a fusion protein compromising 6 histidine residues followed by thioredoxin and an enterokinase cleavage site. The histidine residues facilitate purification on IMIAC (immobilized metal ion affinity chromatography as described in Porath et al (1992) Protein Expression and Purification 3: 263-281), while the enterokinase cleavage site provides a means for separating the hSBP domain from the remainder of the fusion protein.

hSBP polypeptides (which polypeptides encompass polypeptides composed of a portion of the native hSBP amino acid sequence) can also be produced by direct peptide synthesis using solid-phase techniques (cf Stewart et al (1969) Solid-Phase Peptide Synthesis, WH Freeman Co, San Francisco; Merrifield J (1963) J Am Chem Soc 85:2149-2154). In vitro protein synthesis can be performed using manual techniques or by automation. Automated synthesis can be achieved by, for example, using Applied Biosystems 431A Peptide Synthesizer (Perkin Elmer, Foster City Calif.) in accordance with the instructions provided by the manufacturer. Various fragments of hSBP can be chemically synthesized separately and combined using chemical methods to produce the full length molecule.

### Uses of hSBP

The rationale for use of the nucleotide and polypeptide sequences disclosed herein is based in part on the differential expression of hSBP-encoding sequences in breast tumor tissue and in part on the chemical and structural homology between the hSBP proteins disclosed herein and chemical and structural homology between: 1) hSBP1, rat prostatic binding proteins C1 (GI 206442; Delaey et al. supra), rat prostatic binding protein C2(Delaey et al. 1987 Nucl Acid Res 15:1627-1641) and rabbit uteroglobin (Menne et al. 1982 Proc Natl Acad Sci USA 79:4853-4857) (FIG. 5), and 2) hSBP2, human mammaglobin (GI 1199595; Watson et al. supra), and rat prostatic binding protein C3 (GI 206543; Parker et al. supra) (FIG. 6).

Accordingly, hSBP or an hSBP derivative can be used in the diagnosis and management of breast cancer. Given the homology of hSBP with rat PBP, and the differential expression of hSBP in human breast tumor tissue, hSBP can be used as a diagnostic marker for human breast cancer. Expression of rat PBP is regulated by androgens (Muder et al. 1984 Biochem Biophys Acta 781:121-9; Page et al. 1983 Cell 32:495-502) and by growth hormone (Reiter et al. 1995 Endocrinol 166: 3338-44). Thus the level of hSBP can serve as a marker for transformation of normal breast cells into cancerous cells. Alternatively, or in addition, development of breast cancer can be detected by examining the ratio of hSBP to the levels of steroid hormones (e.g., testosterone or estrogen) or to other hormones (e.g., growth hormone, insulin). Thus expression of hSBP1 and/or hSBP2 can also be used to discriminate between normal and cancerous breast tissue, to discriminate between different types of breast cancer, to provide guidance in selection of anti-cancer therapies, to monitor the progress of patients undergoing chemotherapy and/or other anti-cancer treatments, to determine the success of surgery to remove cancerous tissue, and to monitor patients who have had or are susceptible to breast cancer. In addition to diagnosis and treatment of breast cancer after its development, detection of hSBP expression can be used to identify patients susceptible to breast cancer. Expression of hSBP in cancerous cells can be examined in breast tissue in situ or in pathology sections. Alternatively, if hSBP is secreted at sufficient levels, expression of hSBP can be assessed in blood, serum, or plasma. Assessment of levels of hSBP expression can be used to differentiate between normal and cancerous breast tissue, and/or different types of cancerous breast tissue (e.g., invasive vs. non-invasive; ductal vs. axillary lymph node).

In addition, because hSBP is differentially expressed in breast tumor cells, hSBP polypeptides can serve as a target for anti-cancer therapy that is targeted to hSBP-expressing breast tumor cells. For example, cells can be transfected with antisense sequences to hSBP-encoding polynucleotides or provided with antagonists to hSBP to reduce or eliminate hSBP expression in cancerous breast cells. Alternatively, cancerous breast cells, or breast cells susceptible to cancer, can be transformed (e.g., via gene therapy techniques) with hSBP-encoding nucleic acid to provide for expression of excess hSBP and interruption of steroid binding.

### hSBP Antibodies

hSBP-specific antibodies are useful for the diagnosis of conditions and diseases associated with expression of hSBP. Such antibodies include, but are not limited to, polyclonal, monoclonal, chimeric, single chain, Fab fragments and fragments produced by a Fab expression library. Neutralizing antibodies, i.e., those which inhibit a biochemical activity of hSBP, are especially preferred for diagnostics and therapeutics.

hSBP polypeptides suitable for production of antibodies need not be biologically active; rather, the polypeptide, or oligopeptide need only be antigenic. Polypeptides used to generate hSBP-specific antibodies generally have an amino acid sequence consisting of at least five amino acids, preferably at least 10 amino acids. Preferably, antigenic hSBP polypeptides mimic an epitope of the native hSBP. Antibodies specific for short hSBP polypeptides can be generated by linking the hSBP polypeptide to a carrier, or fusing the hSBP polypeptide to another protein (e.g., keyhole limpet hemocyanin), and using the carrier-linked or hSBP chimeric molecule as an antigen. In general, anti-hSBP antibodies can be produced according to methods well known in the art.

Various hosts, generally mammalian hosts, can be used to produce anti-hSBP antibodies (e.g., goats, rabbits, rats, mice). Anti-hSBP antibodies are produced by immunizing the host (e.g., by injection) with an hSBP polypeptide that retains immunogenic properties (which encompasses any portion of native hSBP, fragment or oligopeptide). Depending on the host species, various adjuvants can be used to increase the host's immunological response. Such adjuvants include but are not limited to, Freund's, mineral gels (e.g., aluminum hydroxide), and surface active substances such as lysolecithin, pluronic polyols, polyanions, peptides, oil emulsions, keyhole limpet hemocyanin, and dinitrophenol. BCG (bacilli Calmette-Guerin) and Corynebacterium parvum are potentially useful human adjuvants.

Monoclonal anti-hSBP antibodies can be prepared using any technique that provides for the production of antibody molecules by immortalized cell lines in culture. These techniques include, but are not limited to, the hybridoma technique originally described by Koehler and Milstein (1975 Nature 256:495-497), the human B-cell hybridoma technique (Kosbor et al (1983) Immunol Today 4:72; Cote et al (1983) Proc Natl Acad Sci 80:2026-2030) and the EBV-hybridoma technique (Cole et al (1985) Monoclonal Antibodies and Cancer Therapy, Alan R Liss Inc, New York N.Y., pp 77-96).

In addition, techniques developed for the production of “chimeric antibodies”, the splicing of mouse antibody genes to human antibody genes to obtain a molecule with appropriate antigen specificity and biological activity can be used (Morrison et al (1984) Proc Natl Acad Sci 81:6851-6855; Neuberger et al (1984) Nature 312:604-608; Takeda et al (1985) Nature 314:452-454) Alternatively, techniques described for the production of single chain antibodies (U.S. Pat. No. 4,946,778) can be adapted to produce hSBP-specific single chain antibodies

Antibodies can be produced in vivo or by screening recombinant immunoglobulin libraries or panels of highly specific binding reagents as disclosed in Orlandi et al (1989, Proc Natl Acad Sci 86: 3833-3837), and Winter G and Milstein C (1991; Nature 349:293-299).

Antibody fragments having specific binding sites for an hSBP polypeptide can also be generated. For example, such fragments include, but are not limited to, F(ab′)2 fragments, which can be produced by pepsin digestion of the antibody molecule, and Fab fragments, which can be generated by reducing the disulfide bridges of the F(ab′)2 fragments. Alternatively, Fab expression libraries can be constructed to allow rapid and easy identification of monoclonal Fab fragments with the desired specificity (Huse W D et al (1989) Science 256:1275-1281).

A variety of protocols for competitive binding or immunoradiometric assays using either polyclonal or monoclonal antibodies having established antigen specificities are well known in the art. Such immunoassays typically involve the formation of complexes between an hSBP polypeptide and a specific anti-hSBP antibody, and the detection and quantitation of hSBP-antibody complex formation. A two-site, monoclonal-based immunoassay utilizing monoclonal antibodies reactive to two noninterfering epitopes on a specific hSBP protein is preferred, but a competitive binding assay can also be employed. These assays are described in Maddox D E et al (1983, J Exp Med 158:1211).

### Diagnostic Assays Using hSBP Specific Antibodies

Particular hSBP antibodies are useful for the diagnosis of conditions or diseases characterized by expression of hSBP (e.g., breast cancer) or in assays to monitor patients being treated with hSBP, agonists, antagonists, or inhibitors. Diagnostic assays for hSBP include methods using a detectably-labeled anti-hSBP antibody to detect hSBP in human body fluids or extracts of cells or tissues. The polypeptides and antibodies of the present invention can be used with or without modification. Frequently, the polypeptides and antibodies are labeled by covalent or noncovalent attachment to a reporter molecule. A wide variety of such suitable reporter molecules are known in the art.

A variety of protocols for detection and quantifying hSBP, using either polyclonal or monoclonal antibodies specific for an hSBP polypeptide, are known in the art. Examples include enzyme-linked immunosorbent assay (ELISA), radioimmunoassay (RIA) and fluorescent activated cell sorting (FACS). A two-site, monoclonal-based immunoassay utilizing monoclonal antibodies reactive to two non-interfering epitopes on hSBP is preferred, but a competitive binding assay can instead be employed. These assays are described, among other places, in Maddox, D E et al (1983, J Exp Med 158:1211).

In order to provide a basis for diagnosis, normal or standard values for hSBP expression must be established. This is accomplished by combining body fluids or cell extracts taken from normal subjects, either animal or human, preferably human, with antibody to hSBP under conditions suitable for complex formation according to methods well known in the art. The amount of standard complex formation can be quantified by comparing detection levels associated with known quantities of hSBP with detection levels associated with both control and disease samples from biopsied tissues. Standard values obtained from normal samples are compared with values obtained from samples from subjects potentially affected by disease. Deviation between standard and subject values establishes the presence of disease state.

### Drug Screening

hSBP polypeptides, which encompass biologically active or immunogenic fragments or oligopeptides thereof, can be used for screening therapeutic compounds in any of a variety of drug screening techniques. The polypeptide employed in such a test can be free in solution, affixed to a solid support, borne on a cell surface, or located intracellularly. The formation of binding complexes, between hSBP and the agent being tested, can be measured.

Preferably, the drug screening technique used provides for high throughput screening of compounds having suitable binding affinity to the hSBP, as described in detail in “Determination of Amino Acid Sequence Antigenicity” by Geysen H N, WO Application 84/03564, published on Sep. 13, 1984, and incorporated herein by reference. In summary, large numbers of different small peptide test compounds are synthesized on a solid substrate, such as plastic pins or some other surface. The peptide test compounds are reacted with hSBP polypeptides, unreacted materials are washed away, and bound hSBP is detected by methods well known in the art. Purified hSBP can also be coated directly onto plates for use in the aforementioned drug screening techniques. Alternatively, non-neutralizing antibodies can be used to capture the polypeptide and immobilize it on a solid support.

The invention also contemplates the use of competitive drug screening assays in which hSBP-specific neutralizing antibodies compete with a test compound for binding of hSBP polypeptide. In this manner, the antibodies can be used to detect the presence of any polypeptide that shares one or more antigenic determinants with an hSBP polypeptide.

### Uses of the Polynucleotide Encoding hSBP

A polynucleotide encoding an hSBP polypeptide (which polypeptides include native hSBP and fragments thereof) can be used for diagnostic and/or therapeutic purposes. For diagnostic purposes, polynucleotides encoding hSBP of this invention can be used to detect and quantitate gene expression in biopsied tissues in which expression of hSBP is implicated, particularly in diagnosis of breast cancer. The diagnostic assay is useful to assess hSBP expression levels (e.g., to distinguish between the absence, and presence or hSBP expression, as well as to assess various hSBP expression levels (e.g., excessively high, high, moderate, or low)) and to monitor regulation of hSBP levels during therapeutic intervention. Included in the scope of the invention are oligonucleotide sequences, antisense RNA and DNA molecules, and peptide nucleic acids (PNAs).

Another aspect of the subject invention is to provide for hybridization or PCR probes capable of detecting polynucleotide sequences encoding hSBP, including genomic sequences and closely related molecules. The specificity of the probe, whether it is made from a highly specific region, e.g., unique nucleotides in the 5′ regulatory region, or a less specific region, e.g., especially in the 3′ region, and the stringency of the hybridization or amplification (maximal, high, intermediate or low) will determine whether the probe identifies only naturally occurring sequences encoding hSBP, alleles or related sequences.

The probes of the invention can be used in the detection of related sequences; such probes preferably comprise at least 50% of the nucleotides from any of the hSBP polypeptide-encoding sequences described herein. The hybridization probes of the subject invention can be derived from the nucleotide sequence of SEQ ID NO:2 and SEQ ID NO:4, or from their corresponding genomic sequences including promoters, enhancer elements and introns of the naturally occurring hSBP-encoding sequences. Hybridization probes can be detectably labeled with a variety of reporter molecules, including radionuclides (e.g., 32P or 35S), or enzymatic labels (e.g., alkaline phosphatase coupled to the probe via avidin/biotin coupling systems), and the like.

Specific hybridization probes for hSBP-encoding DNAs can also be produced by cloning nucleic acid sequences encoding hSBP or hSBP derivatives into vectors for production of mRNA probes. Such vectors, which are known in the art and are commercially available, can be used to synthesize RNA probes in vitro using an appropriate RNA polymerase (e.g., T7 or SP6 RNA polymerase) and appropriate radioactively labeled nucleotides.

### Diagnostic Use

Polynucleotide sequences encoding hSBP polypeptide can be used in the diagnosis of conditions or diseases associated with hSBP expression, especially breast cancer. For example, polynucleotide sequences encoding hSBP can be used in hybridization or PCR assays of fluids or tissues from biopsies to detect hSBP expression. Suitable qualitative or quantitative methods include Southern or northern analysis, dot blot or other membrane-based technologies; PCR technologies; dip stick, pIN, chip and ELISA technologies. All of these techniques are well known in the art and are the basis of many commercially available diagnostic kits.

The nucleotide sequences encoding hSBP disclosed herein provide the basis for assays that detect the onset of, susceptibility to, or the presence of breast cancer. Nucleotide sequences encoding hSBP polypeptides can be labeled by methods known in the art and combined with a fluid or tissue sample from a patient suspected of having or susceptible to breast cancer under conditions suitable for the formation of hybridization complexes. After an incubation period, the sample is washed with a compatible fluid which optionally contains a dye (or other label requiring a developer) if the nucleotide has been labeled with an enzyme. After the compatible fluid is rinsed off, the dye is quantitated and compared with a standard. If the amount of dye in the biopsied or extracted sample is significantly elevated over that of a comparable negative control sample, the nucleotide sequence has hybridized with nucleotide sequences in the sample. The presence of hSBP-encoding nucleotide sequences in the sample, particularly the presence of elevated levels of hSBP-encoding sequences, indicates that the patient has or is at risk of developing the associated disease.

Such assays can also be used to evaluate the efficacy of a particular therapeutic treatment regime in animal studies or in clinical trials, or in monitoring the treatment of an individual patient. In order to provide a basis for the diagnosis of disease, a normal or standard profile for hSBP expression must be established. This is accomplished by combining body fluids or cell extracts taken from normal subjects, either animal or human, with hSBP, or a portion thereof, under conditions suitable for hybridization or amplification. Standard hybridization can be quantified by comparing, in the same experiment, the values obtained for normal subjects with those obtained with a dilution series of hSBP containing known amounts of substantially purified hSBP. Standard values obtained from normal samples are compared with values obtained from samples from patients afflicted with hSBP-associated diseases, or suspected of having such diseases (e.g., breast cancer). Deviation between standard and subject values is used to establish the presence of disease.

Once disease is established, a therapeutic agent is administered and a treatment profile is generated. Such assays can be repeated on a regular basis to evaluate whether the values in the profile progress toward or return to a normal or standard pattern of hSBP expression. Successive treatment profiles can be used to show the efficacy of treatment over a period of several days or several months.

Oligonucleotides based upon hSBP sequences can be used in PCR-based techniques, as described in U.S. Pat. Nos. 4,683,195 and 4,965,188. Such oligomers are generally chemically synthesized, or produced enzymatically or by recombinantly. Oligomers generally comprise two nucleotide sequences, one with sense orientation (5′->3′) and one with antisense (3′<-5′), employed under optimized conditions for identification of a specific gene or condition. The same two oligomers, nested sets of oligomers, or even a degenerate pool of oligomers can be employed under less stringent conditions for detection and/or quantitation of closely related DNA or RNA sequences.

Additional methods for quantitation of expression of a particular molecule according to the invention include radiolabeling (Melby P C et al 1993 J Immunol Methods 159:235-44) or biotinylating (Duplaa C et al 1993 Anal Biochem 229-36) nucleotides, coamplification of a control nucleic acid, and interpolation of experimental results according to standard curves. Quantitation of multiple samples can be made more time efficient by running the assay in an ELISA format in which the oligomer of interest is presented in various dilutions and rapid quantitation is accomplished by spectrophotometric or colorimetric detection. For example, the presence of a relatively high amount of hSBP in extracts of biopsied tissues indicates the presence of cancerous breast cells. A definitive diagnosis of this type can allow health professionals to begin aggressive treatment and prevent further worsening of the condition. Similarly, further assays can be used to monitor the progress of a patient during treatment. Furthermore, the nucleotide sequences disclosed herein can be used in molecular biology techniques that have not yet been developed, provided the new techniques rely on properties of nucleotide sequences that are currently known such as the triplet genetic code, specific base pair interactions, and the like.

### Therapeutic Use

Based upon its homology to genes encoding prostatic binding proteins, hSBP polypeptides and its expression profile in breast tumor cells, polynucleotide sequences encoding hSBP disclosed herein may be useful in the treatment of conditions such as breast cancer or other condition associated with hSBP expression or over-expression.

Expression vectors derived from retroviruses, adenovirus, herpes or vaccinia viruses, or from various bacterial plasmids, can be used for delivery of nucleotide sequences to the targeted organ, tissue or cell population. Recombinant vectors for expression of antisense hSBP polynucleotides can be constructed according to methods well known in the art (see, for example, the techniques described in Sambrook et al (supra) and Ausubel et al (supra)).

Polynucleotides comprising the full length cDNA sequence and/or its regulatory elements enable researchers to use sequences encoding hSBP as an investigative tool in sense (Youssoufian H and H F Lodish 1993 Mol Cell Biol 13:98-104) or antisense (Eguchi et al (1991) Ann Rev Biochem 60:631-652) regulation of gene function. Such technology is now well known in the art, and sense or antisense oligomers, or larger fragments, can be designed from various locations along the coding or control regions.

Expression of genes encoding hSBP can be decreased by transfecting a cell or tissue with expression vectors that express high levels of a desired hSBP-encoding fragment. Such constructs can flood cells with untranslatable sense or antisense sequences. Even in the absence of integration into the DNA, such vectors can continue to transcribe RNA molecules until all copies are disabled by endogenous nucleases. Transient expression can last for a month or more with a non-replicating vector (Mettler I, personal communication) and even longer if appropriate replication elements are part of the vector system.

As mentioned above, modifications of gene expression can be obtained by designing antisense molecules, DNA, RNA or PNA, to the control regions of gene encoding hSBP (i.e., the promoters, enhancers, and introns). Oligonucleotides derived from the transcription initiation site, e.g., between −10 and +10 regions of the leader sequence, are preferred. The antisense molecules can also be designed to block translation of mRNA by preventing the transcript from binding to ribosomes. Similarly, inhibition of expression can be achieved using “triple helix” base-pairing methodology. Triple helix pairing compromises the ability of the double helix to open sufficiently for binding of polymerases, transcription factors, or regulatory molecules. Recent therapeutic advances using triplex DNA were reviewed by Gee J E et al (In: Huber B E and B I Carr (1994) Molecular and Immunologic Approaches, Futura Publishing Co, Mt Kisco N.Y.).

Ribozymes are enzymatic RNA molecules capable of catalyzing the specific cleavage of RNA. The mechanism of ribozyme action involves sequence-specific hybridization of the ribozyme molecule to complementary target RNA, followed by endonucleolytic cleavage. The invention contemplates engineered hammerhead motif ribozyme molecules that can specifically and efficiently catalyze endonucleolytic cleavage of sequences encoding hSBP.

Specific ribozyme cleavage sites within any potential RNA target are initially identified by scanning the target molecule for ribozyme cleavage sites, which sites include the following sequences, GUA, GUU and GUC. Once identified, short RNA sequences between 15 and 20 ribonucleotides corresponding to a region of the target gene containing the cleavage site can be evaluated for secondary structural features that can render the oligonucleotide inoperable. The suitability of candidate targets can also be evaluated by testing accessibility to hybridization with complementary oligonucleotides using ribonuclease protection assays.

Antisense molecules and ribozymes of the invention can be prepared by methods known in the art for the synthesis of RNA molecules, including techniques for chemical oligonucleotide synthesis, e.g., solid phase phosphoramidite chemical synthesis. Alternatively, RNA molecules can be generated by in vitro and in vivo transcription of DNA sequences encoding hSBP. Such DNA sequences can be incorporated into a wide variety of vectors with suitable RNA polymerase promoters (e.g., T7 or SP6). Alternatively, antisense cDNA constructs useful in the constitutive or inducible synthesis of antisense RNA can be introduced into cell lines, cells, or tissues.

RNA molecules can be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine and wybutosine as well as acetyl-, methyl-, thio- and similarly modified forms of adenine, cytidine, guanine, thymine, and uridine that are not as easily recognized by endogenous endonucleases.

Methods for introducing vectors into cells or tissues include those methods discussed infra and which are equally suitable for in vivo, in vitro and ex vivo therapy. In ex vivo therapy, vectors are introduced into stem cells obtained from the patient and clonally propagated for autologous transplant back into that same patient (see, e.g., U.S. Pat. Nos. 5,399,493 and 5,437,994, incorporated herein by reference). Transfection and by liposome methods for delivery of a nucleotide sequence of interest to accomplish gene therapy are well known in the art.

Furthermore, the nucleotide sequences for hSBP disclosed herein can be used in molecular biology techniques that have not yet been developed, provided the new techniques rely on properties of nucleotide sequences that are currently known, including but not limited to such properties as the triplet genetic code and specific base pair interactions.

### Detection and Mapping of Related Polynucleotide Sequences

The hSBP nucleic acid sequences can also be used to generate hybridization probes for mapping the naturally occurring genomic sequence. The sequence can be mapped to a particular chromosome or to a specific region of the chromosome using well known techniques. These include in situ hybridization to chromosomal spreads, flow-sorted chromosomal preparations, or artificial chromosome constructions such as yeast artificial chromosomes, bacterial artificial chromosomes, bacterial P1 constructions or single chromosome cDNA libraries as reviewed in Price C M (1993; Blood Rev 7:127-34) and Trask B J (1991; Trends Genet 7:149-54).

The technique of fluorescent in situ hybridization of chromosome spreads is described in, for example, Verma et al (1988) Human Chromosomes: A Manual of Basic Techniques, Pergamon Press, New York N.Y. Fluorescent in situ hybridization of chromosomal preparations and other physical chromosome mapping techniques can be correlated with additional genetic map data. Examples of genetic map data can be found in the 1994 Genome Issue of Science (265:1981f). Correlation between the location of a gene encoding hSBP on a physical chromosomal map and a specific disease (or predisposition to a specific disease) can help delimit the region of DNA associated with that genetic disease. The nucleotide sequences of the subject invention can be used to detect differences in gene sequences between normal, carrier, or affected individuals.

In situ hybridization of chromosomal preparations and physical mapping techniques such as linkage analysis using established chromosomal markers can be used for extending genetic maps. For example an sequence tagged site based map of the human genome was recently published by the Whitehead-MIT Center for Genomic Research (Hudson T J et al (1995) Science 270:1945-1954). Often the placement of a gene on the chromosome of another mammalian species such as a mouse (Whitehead Institute/MIT Center for Genome Research, Genetic Map of the Mouse, Database Release 10, Apr. 28, 1995) can reveal associated markers even if the number or arm of a particular human chromosome is not known. New sequences can be assigned to chromosomal arms, or parts thereof, by physical mapping. Physical mapping provides valuable information to investigators searching for disease genes using positional cloning or other gene discovery techniques. Once a disease or syndrome, such as ataxia telangiectasia (AT), has been crudely localized by genetic linkage to a particular genomic region, for example, AT to 11q22-23 (Gatti et al (1988) Nature 336:577-580), other sequences mapping to that area may represent associated or regulatory genes for further investigation. The nucleotide sequence of the subject invention can also be used to detect differences in the chromosomal location due to translocation, inversion, etc. among normal, carrier or affected individuals.

### Pharmaceutical Compositions

The present invention relates to pharmaceutical compositions which can comprise nucleotides, proteins, antibodies, agonists, antagonists, or inhibitors, alone or in combination with at least one other agent, such as a stabilizing compound, which can be administered in any sterile, biocompatible pharmaceutical carrier, including, but not limited to, saline, buffered saline, dextrose, and water. Any of these molecules can be administered to a patient alone or in combination with other agents, drugs or hormones, in pharmaceutical compositions where it is mixed with excipient(s), or with pharmaceutically acceptable carriers. In one embodiment of the present invention, the pharmaceutically acceptable carrier is pharmaceutically inert.

### Administration of Pharmaceutical Compositions

Administration of pharmaceutical compositions is accomplished orally or parenterally. Methods of parenteral delivery include topical, intra-arterial (e.g., directly to the breast tumor), intramuscular, subcutaneous, intramedullary, intrathecal, intraventricular, intravenous, intraperitoneal, or intranasal administration. In addition to the active ingredients, these pharmaceutical compositions can contain suitable pharmaceutically acceptable carriers comprising excipients and auxiliaries that facilitate processing of the active compounds into preparations for pharmaceutical use. Further details on techniques for formulation and administration can be found in the latest edition of “Remington's Pharmaceutical Sciences” (Maack Publishing Co, Easton Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

Pharmaceutical preparations for oral use can be obtained through combination of active compounds with solid excipient, optionally grinding a resulting mixture, and processing the mixture of granules, after adding suitable auxiliaries if desired, to obtain tablets or dragee cores. Suitable excipients are carbohydrate or protein fillers such as sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; and proteins such as gelatin and collagen. If desired, disintegrating or solubilizing agents can be added, such as the cross-linked polyvinyl pyrrolidone, agar, alginic acid, or a salt thereof, such as sodium alginate.

Dragee cores are provided with suitable coatings such as concentrated sugar solutions, which can also contain gum arabic, talc, polyvinylpyrrolidone, carbopol gel, polyethylene glycol, and/or titanium dioxide, lacquer solutions, and suitable organic solvents or solvent mixtures. Dyestuffs or pigments can be added to the tablets or dragee coatings for product identification or to characterize the quantity of active compound, i.e., dosage.

Pharmaceutical preparations that can be used orally include push-fit capsules made of gelatin, as well as soft, sealed capsules made of gelatin and a coating such as glycerol or sorbitol. Push-fit capsules can contain active ingredients mixed with a filler or binders such as lactose or starches, lubricants such as talc or magnesium stearate, and, optionally, stabilizers. In soft capsules, the active compounds can be dissolved or suspended in suitable liquids, such as fatty oils, liquid paraffin, or liquid polyethylene glycol with or without stabilizers.

Pharmaceutical formulations for parenteral administration include aqueous solutions of active compounds. For injection, the pharmaceutical compositions of the invention can be formulated in aqueous solutions, preferably in physiologically compatible buffers such as Hanks's solution, Ringer's solution, or physiologically buffered saline. Aqueous injection suspensions can contain substances that increase the viscosity of the suspension, such as sodium carboxymethyl cellulose, sorbitol, or dextran. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension can also contain suitable stabilizers or agents that increase the solubility of the compounds to allow for the preparation of highly concentrated solutions.

For topical or nasal administration, penetrants appropriate to the particular barrier to be permeated are used in the formulation. Such penetrants are generally known in the art.

### Manufacture and Storage

The pharmaceutical compositions of the present invention can be manufactured in any suitable manner known in the art, e.g., by means of conventional mixing, dissolving, granulating, dragee-making, levigating, emulsifying, encapsulating, entrapping or lyophilizing processes.

The pharmaceutical composition can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preferred preparation can be a lyophilized powder in 1 mM-50 mM histidine, 0.1%-2% sucrose, 2%-7% mannitol at a pH range of 4.5 to 5.5 that is combined with buffer prior to use.

After pharmaceutical compositions comprising a compound of the invention formulated in a acceptable carrier have been prepared, they can be placed in an appropriate container and labeled for treatment of an indicated condition. For administration of hSBP, such labeling would include amount, frequency and method of administration.

### Therapeutically Effective Dose

Pharmaceutical compositions suitable for use in the present invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, e.g., of neoplastic cells, or in animal models, usually mice, rabbits, dogs, or pigs. The animal model is also used to achieve a desirable concentration range and route of administration. Such information can then be used to determine useful doses and routes for administration in humans.

A therapeutically effective dose refers to that amount of protein or its antibodies, antagonists, or inhibitors that ameliorate the symptoms or condition. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and expressed as the ratio LD50/ED50. Pharmaceutical compositions that exhibit large therapeutic indices are preferred. The data obtained from cell culture assays and animal studies is used in formulating a range of dosage for human use. The dosage of such compounds lies preferably within a range of circulating concentrations that include the ED50 with little or no toxicity. The actual dosage can vary within this range depending upon, for example, the dosage form employed, sensitivity of the patient, and the route of administration.

The exact dosage is chosen by the individual physician in view of the patient to be treated. Dosage and administration are adjusted to provide sufficient levels of the active moiety or to maintain the desired effect. Additional factors that may be taken into account include the severity of the disease state, e.g., tumor size and location; age, weight and gender of the patient; diet, time and frequency of administration; drug combination(s); reaction sensitivities; and tolerance/response to therapy. Long-acting pharmaceutical compositions can be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

It is contemplated, for example, that hSBP or an hSBP derivative can be delivered in a suitable formulation to block the progression of breast cancer. Similarly, administration of hSBP antagonists may also inhibit the activity or shorten the lifespan of this protein.

The examples below are provided to illustrate the subject invention and are not included for the purpose of limiting the invention.

### EXAMPLES

I. Construction of BRSTTUT01 cDNA Libraries

The BRSTTUT01 cDNA library was constructed from breast tumor removed from a 55 year old female. The frozen tissue was immediately homogenized and lysed using a Brinkmann Homogenizer Polytron-PT 3000 (Brinkmann Instruments, Inc. Westbury N.Y.) in guanidinium isothiocyanate solution. Lysates were then loaded on a 5.7 M CsCl cushion and ultracentrifuged in a SW28 swinging bucket rotor for 18 hours at 25,000 rpm at ambient temperature. The RNA was extracted once with acid phenol at pH 4.0 and once with phenol chloroform at pH 8.0 and precipitated using 0.3 M sodium acetate and 2.5 volumes of ethanol, resuspended in DEPC-treated water and DNase treated for 25 min at 37°. The reaction was stopped with an equal volume of acid phenol, and the RNA was isolated using the Qiagen Oligotex kit (QIAGEN Inc, Chatsworth Calif.) and used to construct the cDNA library. The RNA was handled according to the recommended protocols in the SuperScript Plasmid System for cDNA Synthesis and Plasmid Cloning (catalog # 18248-013; Gibco/BRL). cDNAs were fractionated on a Sepharose CL4B column (catalog # 275105, Pharmacia), and those cDNAs exceeding 400 bp were ligated into pSport I. The plasmid pSport I was subsequently transformed into DH5a(tm) competent cells (Cat. # 18258-012, Gibco/BRL).

II. Isolation and Sequencing of cDNA Clones From BRSTTUT01

Plasmid DNA was released from the cells and purified using the Miniprep Kit (Catalogue # 77468; Advanced Genetic Technologies Corporation, Gaithersburg Md.). This kit consists of a 96 well block with reagents for 960 purifications. The recommended protocol was employed except for the following changes: 1) the 96 wells were each filled with only 1 ml of sterile Terrific Broth (Catalog # 22711, LIFE TECHNOLOGIES(tm), Gaithersburg Md.) with carbenicillin at 25 mg/L and glycerol at 0.4%; 2) the bacteria were cultured for 24 hours after the wells were inoculated and then lysed with 60 μl of lysis buffer; 3) a centrifugation step employing the Beckman GS-6R @2900 rpm for 5 min was performed before the contents of the block were added to the primary filter plate; and 4) the optional step of adding isopropanol to TRIS buffer was not routinely performed. After the last step in the protocol, samples were transferred to a Beckman 96-well block for storage.

The cDNAs were sequenced by the method of Sanger F and A R Coulson (1975; J Mol Biol 94:441f), using a Hamilton Micro Lab 2200 (Hamilton, Reno Nev.) in combination with four Peltier Thermal Cyclers (PTC200 from MJ Research, Watertown Mass.) and Applied Biosystems 377 or 373 DNA Sequencing Systems (Perkin Elmer), and reading frame was determined.

III. Homology Searching of cDNA Clones and Their Deduced Proteins

Each cDNA was compared to sequences in GenBank using a search algorithm developed by Applied Biosystems and incorporated into the INHERIT™ 670 Sequence Analysis System. In this algorithm, Pattern Specification Language (TRW Inc, Los Angeles Calif.) was used to determine regions of homology. The three parameters that determine how the sequence comparisons run were window size, window offset, and error tolerance. Using a combination of these three parameters, the DNA database was searched for sequences containing regions of homology to the query sequence, and the appropriate sequences were scored with an initial value. Subsequently, these homologous regions were examined using dot matrix homology plots to distinguish regions of homology from chance matches. Smith-Waterman alignments were used to display the results of the homology search.

Peptide and protein sequence homologies were ascertained using the INHERIT-670 Sequence Analysis System in a way similar to that used in DNA sequence homologies. Pattern Specification Language and parameter windows were used to search protein databases for sequences containing regions of homology which were scored with an initial value. Dot-matrix homology plots were examined to distinguish regions of significant homology from chance matches.

BLAST, which stands for Basic Local Alignment Search Tool (Altschul S F (1993) J Mol Evol 36:290-300; Altschul, S F et al (1990) J Mol Biol 215:403-10), was used to search for local sequence alignments. BLAST produces alignments of both nucleotide and amino acid sequences to determine sequence similarity. Because of the local nature of the alignments, BLAST is especially useful in determining exact matches or in identifying homologs. BLAST is useful for matches that do not contain gaps. The fundamental unit of BLAST algorithm output is the High-scoring Segment Pair (HSP).

An HSP consists of two sequence fragments of arbitrary but equal lengths whose alignment is locally maximal and for which the alignment score meets or exceeds a threshold or cutoff score set by the user. The BLAST approach identifies HSPs between a query sequence and a database sequence, evaluates the statistical significance of any matches found, and reports only those matches which satisfy the user-selected threshold of significance. The parameter E establishes the statistically significant threshold for reporting database sequence matches. E is interpreted as the upper bound of the expected frequency of chance occurrence of an HSP (or set of HSPs) within the context of the entire database search. Any database sequence whose match satisfies E is reported in the program output.

IV. Northern Analysis

Northern analysis, a laboratory technique used to detect the presence of a gene transcript, and involves the hybridization of a labeled nucleotide sequence to a membrane on which RNAs from a particular cell type or tissue have been bound (Sambrook et al. supra).

Analogous computer techniques using BLAST (Altschul S F 1993 and 1990, supra) are used to search for identical or related molecules in nucleotide databases such as GenBank or the LIFESEQ™ database (Incyte, Palo Alto Calif.). This analysis is much faster than multiple, membrane-based hybridizations. In addition, the sensitivity of the computer search can be modified to determine whether any particular match is categorized as exact or homologous.

The basis of the search is the product score which is defined as:

$$\frac{\% sequence identity \times \% maximum BLAST score}{100}$$

The product score takes into account both the degree of similarity between two sequences and the length of the sequence match. For example, with a product score of 40, the match will be exact within a 1-2% error; and at 70, the match will be exact. Homologous molecules are usually identified by selecting those which show product scores between 15 and 40, although lower scores can identify related molecules. The abundance data (Abun) represent the number of transcripts of the gene of interest in the cDNA library. Percent abundance is calculated by dividing the number of transcripts of a gene of interest present in a cDNA library by the total number of transcripts in the cDNA library.

V. Extension of hSBP-Encoding Polynucleotides to Full Length or to Recover Regulatory Elements

Full length hSBP-encoding nucleic acid sequences (SEQ ID NO:2, SEQ ID NO:4, or SEQ ID NO:6) are used to design oligonucleotide primers for extending a partial nucleotide sequence to full length and/or for obtaining 5′ sequences from genomic libraries. One synthesized primer is used to initiate extension in the antisense direction (XLR), and a second synthesized primer is used to extend sequence in the sense direction (XLF). Primers allow the extension of the known hSBP-encoding sequence “outward” generating amplicons containing new, unknown nucleotide sequence for the region of interest (U.S. patent application Ser. No. 08/487,112, filed Jun. 7, 1995, specifically incorporated by reference). The initial primers are designed from the cDNA using OLIGO® 4.06 Primer Analysis Software (National Biosciences), or another appropriate program. The initial primers are preferable designed to be 22-30 nucleotides in length, have a GC content of 50% or more, and to anneal to the target sequence at temperatures about 68°-72° C. Any stretch of nucleotides that would result in hairpin structures and primer-primer dimerizations is avoided.

The original, selected cDNA libraries, or a human genomic library, are used to extend the sequence; the latter is most useful to obtain 5′ upstream regions. If more extension is necessary or desired, additional sets of primers are designed to further extend the known region.

By following the instructions for the XL-PCR kit (Perkin Elmer) and thoroughly mixing the enzyme and reaction mix, high fidelity amplification is obtained. Beginning with 40 pmol of each primer and the recommended concentrations of all other components of the kit, PCR is performed using the Peltier Thermal Cycler (PTC200; MJ Research, Watertown Mass.) and the following parameters:

| | |
|---|---|
| Step 1 | 94° C. for 1 min (initial denaturation) |
| Step 2 | 65° C. for 1 min |
| Step 3 | 68° C. for 6 min |
| Step 4 | 94° C. for 15 sec |
| Step 5 | 65° C. for 1 min |
| Step 6 | 68° C. for 7 min |
| Step 7 | Repeat step 4-6 for 15 additional cycles |
| Step 8 | 94° C. for 15 sec |
| Step 9 | 65° C. for 1 min |
| Step 10 | 68° C. for 7:15 min |
| Step 11 | Repeat step 8-10 for 12 cycles |
| Step 12 | 72° C. for 8 min |
| Step 13 | 4° C. (and holding) |

A 5-10 μl aliquot of the reaction mixture is analyzed by electrophoresis on a low concentration (about 0.6-0.8%) agarose mini-gel to determine which reactions were successful in extending the sequence. Bands containing the largest products were selected and cut out of the gel. Further purification is accomplished using a commercial gel extraction method such as QIAQuick™ (QIAGEN Inc). After recovery of the DNA, Klenow enzyme was used to trim single-stranded, nucleotide overhangs creating blunt ends to facilitate religation and cloning.

After ethanol precipitation, the products are redissolved in 13 μl of ligation buffer, 1 μl T4-DNA ligase (15 units) and 1 μl T4 polynucleotide kinase are added, and the mixture is incubated at room temperature for 2-3 hours or overnight at 16° C. Competent E. coli cells (in 40 μl of appropriate media) are transformed with 3 μl of ligation mixture and cultured in 80 μl of SOC medium (Sambrook J et al, supra). After incubation for one hour at 37° C., the whole transformation mixture is plated on Luria Bertani (LB)-agar (Sambrook J et al, supra) containing 2×Carb. The following day, several colonies are randomly picked from each plate and cultured in 150 μl of liquid LB/2×Carb medium placed in an individual well of an appropriate, commercially-available, sterile 96-well microtiter plate. The following day, 5 μl of each overnight culture is transferred into a non-sterile 96-well plate and after dilution 1:10 with water, 5 μl of each sample was transferred into a PCR array.

For PCR amplification, 18 μl of concentrated PCR reaction mix (3.3×) containing 4 units of rTth DNA polymerase, a vector primer and one or both of the gene specific primers used for the extension reaction were added to each well. Amplification was performed using the following conditions:

| | |
|---|---|
| Step 1 | 94° C. for 60 sec |
| Step 2 | 94° C. for 20 sec |
| Step 3 | 55° C. for 30 sec |
| Step 4 | 72° C. for 90 sec |
| Step 5 | Repeat steps 2-4 for an additional 29 |
| | cycles |
| Step 6 | 72° C. for 180 sec |
| Step 7 | 4° C. (and holding) |

Aliquots of the PCR reactions are run on agarose gels together with molecular weight markers. The sizes of the PCR products were compared to the original partial cDNAs, and appropriate clones were selected, ligated into plasmid and sequenced.

VI. Labeling and Use of Hybridization Probes

Hybridization probes derived from SEQ ID NO:2 and SEQ ID NO:4 are used to screen cDNAs, genomic DNAs or mRNAs. Although the labeling of oligonucleotides, consisting of about 20 base-pairs, is specifically described, essentially the same procedure is used with larger cDNA fragments. Oligonucleotides are designed using state-of-the-art software such as OLIGO 4.06 (National Biosciences), labeled by combining 50 pmol of each oligomer and 250 mCi of [γ-³²P] adenosine triphosphate (Amersham, Chicago Ill.) and T4 polynucleotide kinase (DuPont NEN®, Boston Mass.). The labeled oligonucleotides are substantially purified with Sephadex G-25 super fine resin column (Pharmacia). A portion containing 10⁷ counts per minute of each of the sense and antisense oligonucleotides is used in a typical membrane based hybridization analysis of human genomic DNA digested with one of the following endonucleases (Ase I, Bgl II, Eco RI, Pst I, Xba 1, or Pvu II; DuPont NEN®).

The DNA from each digest is fractionated on a 0.7 percent agarose gel and transferred to nylon membranes (Nytran Plus, Schleicher & Schuell, Durham N.H.). Hybridization is carried out for 16 hours at 40° C. To remove nonspecific signals, blots are sequentially washed at room temperature under increasingly stringent conditions up to 0.1×saline sodium citrate and 0.5% sodium dodecyl sulfate. After XOMAT AR™ film (Kodak, Rochester N.Y.) is exposed to the blots, or the blots are exposed in a PhosphorImager cassette (Molecular Dynamics, Sunnyvale Calif.), hybridization patterns are compared visually.

VII. Antisense Molecules

An hSBP polypeptide-encoding sequence (which sequences encompass full length and partial hSBP sequences), is used to inhibit in vivo or in vitro expression of naturally occurring hSBP. Although use of antisense oligonucleotides, comprising about 20 base-pairs, is specifically described, essentially the same procedure is used with larger cDNA fragments. An oligonucleotide based on the coding sequences of hSBP, as shown in FIG. 1 and FIGS. 2A and 2B is used to inhibit expression of naturally occurring hSBP. The complementary oligonucleotide is designed from the most unique 5′ sequence as shown in FIG. 1 and FIGS. 2A and 2B and used either to inhibit transcription by preventing promoter binding to the upstream nontranslated sequence or translation of an hSBP-encoding transcript by preventing the ribosome from binding. Using an appropriate portion of the leader and 5′ sequence of SEQ ID NO:2 or SEQ ID NO:4, an effective antisense oligonucleotide includes any 15-20 nucleotides spanning the region which translates into the signal or early coding sequence of the polypeptide as shown in FIG. 1 and FIGS. 2A and 2B.

VIII. Expression of hSBP

Expression of the hSBP is accomplished by subcloning the cDNAs into appropriate vectors and transfecting the vectors into host cells. In this case, the cloning vector, pSport, previously used for the generation of the cDNA library is used to express hSBP polypeptides in E. coli. The pSport vector contains a promoter for β-galactosidase upstream of the cloning site, followed by a sequence encoding the amino-terminal Met and the subsequent 7 residues of β-galactosidase. Sequences encoding a bacteriophage promoter useful for transcription and a linker containing a number of unique restriction sites are positioned immediately after the eight β-galactosidase residue-encoding sequences.

IPTG is used to induce production of the fusion protein in an isolated, transfected bacterial strain according to standard methods. The fusion protein comprises the first seven residues of β-galactosidase, about 5 to 15 residues of linker, and the full length hSBP-encoding sequence. The signal sequence directs the secretion of hSBP polypeptide into the bacterial growth media, which can then be used directly in the following activity assay.

IX. hSBP Activity

Given the homology of hSBP with rat prostatic binding protein (rPBP), human mammaglobin, rabbit uteroglobin, and FHG 22, activity of hSBP can be assessed by the ability of the polypeptide to bind to steroid. Methods for assessing steroid binding to a polypeptide are well known in the art (see, e.g., Heyns et al. 1977 Eur J Biochem 78:221-230). Alternatively, given the homology between hSBP and rPBP, and the similarities between rPBP and estramucine binding protein (EMBP), hSBP activity can be assessed by the ability of hSBP to bind estrmucine. Methods for assessing estramucine binding are well known in the art (see, e.g., Appelgren et al. 1979 Acta Pharmacol Toxicol 43:368-374; Forsgren et al. 1979 Cancer Res 39:5155-5164; Høisaeter et al. 1981 J Steroid Biochem 14:251-160).

X. Production of hSBP Specific Antibodies

hSBP polypeptide substantially purified using PAGE electrophoresis (Sambrook, supra) is used to immunize-rabbits and to produce antibodies using standard protocols. The amino acid sequence translated from hSBP is analyzed using DNAStar software (DNAStar Inc) to determine regions of high immunogenicity, and a corresponding oligopolypeptide is synthesized and used to produce antibodies according to methods known to those of skill in the art. Analysis to select appropriate epitopes, such as those near the C-terminus or in hydrophilic regions is described by Ausubel et al (supra).

Typically, antibodies are generated using polypeptides about 15 residues in length, which are synthesized on an Applied Biosystems Peptide Synthesizer Model 431A using fmoc-chemistry, and coupled to keyhole limpet hemocyanin (KLH, Sigma) by reaction with M-maleimidobenzoyl-N-hydroxysuccinimide ester (MBS; Ausubel et al, supra). Rabbits are immunized with the polypeptide-KLH complex in complete Freund's adjuvant. The resulting antisera are tested for anti-polypeptide activity by, for example, binding the peptide to plastic, blocking with 1% BSA, reacting with rabbit antisera, washing, and reacting with radioiodinated, goat anti-rabbit IgG.

XI. Purification of Naturally Occurring hSBP Using Specific Antibodies

Naturally-occurring or recombinant hSBP is substantially purified by immunoaffinity chromatography using antibodies specific for hSBP. An immunoaffinity column is constructed by covalently coupling anti-hSBP antibody to an activated chromatographic resin such as CNBr-activated Sepharose (Pharmacia Biotech). After coupling, the resin is blocked and washed according to the manufacturer's instructions.

Media containing hSBP polypeptide is passed over the immunoaffinity column, and the column is washed under conditions that allow the preferential absorbance of hSBP (e.g., high ionic strength buffers in the presence of detergent). The column is eluted under conditions that disrupt antibody-hSBP binding (e.g., a buffer of pH 2-3 or a high concentration of a chaotrope such as urea or thiocyanate ion), and hSBP polypeptide is collected.

XII. Identification of Molecules Which Interact with hSBP

hSBP polypeptides, especially biologically active hSBP polypeptides, are labeled with ¹²⁵I Bolton-Hunter reagent (Bolton and Hunter (1973) Biochem J 133: 529). Candidate molecules previously arrayed in the wells of a 96 well plate are incubated with the labeled hSBP polypeptides, washed, and assayed for labeled hSBP complex. Data obtained using different concentrations of hSBP are used to calculate values for the number, affinity, and association of hSBP with the candidate molecules.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

Before the present nucleotide and polypeptide sequences are described, it is to be understood that this invention is not limited to the particular methodology, protocols, cell lines, vectors and reagents described as such may, of course, vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “and”, and “the” include plural referents unless the context clearly dictates otherwise. Thus, for example, reference to “a host cell” includes a plurality of such host cells and reference to “the antibody” includes reference to one or more antibodies and equivalents thereof known to those skilled in the art, and so forth.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the cell lines, vectors, and methodologies which are described in the publications which might be used in connection with the presently described invention. The publications discussed herein are provided solely for their disclosure prior to the filing date of the present application. Nothing herein is to be construed as an admission that the inventors are not entitled to antedate such disclosure by virtue of prior invention.

```
# SEQUENCE LIS
#TING
(1) GENERAL INFORMATION:
      (iii) NUMBER OF SEQUENCES: 13
(2) INFORMATION FOR SEQ ID NO: 1:
          (i) SEQUENCE CHARACTERISTICS:
                    (A) LENGTH: 90 amino
#acids
                    (B) TYPE: amino acid
                    (C) STRANDEDNESS: double
                    (D) TOPOLOGY: linear
        (ii) MOLECULE TYPE: <Unknown>
        (xi) SEQUENCE DESCRIPTION: SEQ ID NO:
#1:
Met Lys Leu Ser Val Cys Leu Leu Leu Val Th
#r Leu Ala Leu Cys Cys
1 5
# 10
# 15
Tyr Gln Ala Asn Ala Glu Phe Cys Pro Ala Le
#u Val Ser Glu Leu Leu
                        20
# 25
# 30
Asp Phe Phe Phe Ile Ser Glu Pro Leu Phe Ly
#s Leu Ser Leu Ala Lys
                35
# 40
# 45
Phe Asp Ala Pro Pro Glu Ala Val Ala Ala Ly
#s Leu Gly Val Lys Arg
        50
# 55
# 60
Cys Thr Asp Gln Met Ser Leu Gln Lys Arg Se
#r Leu Ile Ala Glu Val
65
#70
#75
#80
Leu Val Lys Ile Leu Lys Lys Cys Ser Val
                                85
# 90
(2) INFORMATION FOR SEQ ID NO: 2:
          (i) SEQUENCE CHARACTERISTICS:
                    (A) LENGTH: 405 base
#pairs
                    (B) TYPE: nucleic acid
                    (C) STRANDEDNESS: double
                    (D) TOPOLOGY: linear
        (ii) MOLECULE TYPE: cDNA
        (xi) SEQUENCE DESCRIPTION: SEQ ID NO:
#2:
GTCCAAATCA CTCATTGTTT GTGAAAGCTG AGCTCACAGC AAAACAAGCC AC
#C ATG 56
#
#
# Met
#
#
# 1
AAG CTG TCG GTG TGT CTC CTG CTG GTC ACG CT
#G GCC CTC TGC TGC TAC 104
Lys Leu Ser Val Cys Leu Leu Leu Val Thr Le
#u Ala Leu Cys Cys Tyr
                            5
# 10
# 15
CAG GCC AAT GCC GAG TTC TGC CCA GCT CTT GT
#T TCT GAG CTG TTA GAC 152
Gln Ala Asn Ala Glu Phe Cys Pro Ala Leu Va
#l Ser Glu Leu Leu Asp
                  20
# 25
# 30
TTC TTC TTC ATT AGT GAA CCT CTG TTC AAG TT
#A AGT CTT GCC AAA TTT 200
Phe Phe Phe Ile Ser Glu Pro Leu Phe Lys Le
#u Ser Leu Ala Lys Phe
          35
# 40
# 45
GAT GCC CCT CCG GAA GCT GTT GCA GCC AAG TT
#A GGA GTG AAG AGA TGC 248
Asp Ala Pro Pro Glu Ala Val Ala Ala Lys Le
#u Gly Val Lys Arg Cys
  50
# 55
# 60
# 65
ACG GAT CAG ATG TCC CTT CAG AAA CGA AGC CT
#C ATT GCG GAA GTC CTG 296
Thr Asp Gln Met Ser Leu Gln Lys Arg Ser Le
#u Ile Ala Glu Val Leu
                                  70
# 75
# 80
GTG AAA ATA TTG AAG AAA TGT AGT GTG TGA CA
#TGTAAAAA CTTTCATCCT 346
Val Lys Ile Leu Lys Lys Cys Ser Val *
                          85
# 90
GGTTTCCACT GTCTTTCAAT GACACCCTGA TCTTCACTGC AGAATGTAAA GG
#TTTCAAC 405
(2) INFORMATION FOR SEQ ID NO: 3:
          (i) SEQUENCE CHARACTERISTICS:
                    (A) LENGTH: 93 amino
#acids
                    (B) TYPE: amino acid
                    (C) STRANDEDNESS: double
                    (D) TOPOLOGY: linear
        (ii) MOLECULE TYPE: <Unknown>
        (xi) SEQUENCE DESCRIPTION: SEQ ID NO:
#3:
Met Lys Leu Leu Met Val Leu Met Leu Ala Al
#a Leu Ser Gln His Cys
1 5
# 10
# 15
Tyr Ala Gly Ser Gly Cys Pro Leu Leu Glu As
#n Val Ile Ser Lys Thr
                        20
# 25
# 30
Ile Asn Pro Gln Val Ser Lys Thr Glu Tyr Ly
#s Glu Leu Leu Gln Glu
                35
# 40
# 45
Phe Ile Asp Asp Asn Ala Thr Thr Asn Ala Il
#e Asp Glu Leu Lys Glu
        50
# 55
# 60
Cys Phe Leu Asn Gln Thr Asp Glu Thr Leu Se
#r Asn Val Glu Val Phe
65
#70
#75
#80
Met Gln Leu Ile Tyr Asp Ser Ser Leu Cys As
#p Leu Phe
                                85
# 90
(2) INFORMATION FOR SEQ ID NO: 4:
          (i) SEQUENCE CHARACTERISTICS:
                    (A) LENGTH: 495 base
#pairs
                    (B) TYPE: nucleic acid
                    (C) STRANDEDNESS: double
                    (D) TOPOLOGY: linear
        (ii) MOLECULE TYPE: cDNA
        (xi) SEQUENCE DESCRIPTION: SEQ ID NO:
#4:
GATCCTTGCC ACCCGCGACT GAACACCGAC AGCAGCAGCC TCACC ATG
#AAG TTG 54
#
# Met Lys L
#eu
#
# 1
CTG ATG GTC CTC ATG CTG GCG GCC CTC TCC CA
#G CAC TGC TAC GCA GGC 102
Leu Met Val Leu Met Leu Ala Ala Leu Ser Gl
#n His Cys Tyr Ala Gly
            5
# 10
# 15
TCT GGC TGC CCC TTA TTG GAG AAT GTG ATT TC
#C AAG ACA ATC AAT CCA 150
Ser Gly Cys Pro Leu Leu Glu Asn Val Ile Se
#r Lys Thr Ile Asn Pro
  20
# 25
# 30
# 35
CAA GTG TCT AAG ACT GAA TAC AAA GAA CTT CT
#T CAA GAG TTC ATA GAC 198
Gln Val Ser Lys Thr Glu Tyr Lys Glu Leu Le
#u Gln Glu Phe Ile Asp
                                  40
# 45
# 50
GAC AAT GCC ACT ACA AAT GCC ATA GAT GAA TT
#G AAG GAA TGT TTT CTT 246
Asp Asn Ala Thr Thr Asn Ala Ile Asp Glu Le
#u Lys Glu Cys Phe Leu
                          55
# 60
# 65
AAC CAA ACG GAT GAA ACT CTG AGC AAT GTT GA
#G GTG TTT ATG CAA TTA 294
Asn Gln Thr Asp Glu Thr Leu Ser Asn Val Gl
#u Val Phe Met Gln Leu
                  70
# 75
# 80
ATA TAT GAC AGC AGT CTT TGT GAT TTA TTT TA
#A CTT TCT GCA AGA CCT 342
Ile Tyr Asp Ser Ser Leu Cys Asp Leu Phe
#*
          85
# 90
TTG GCT CAC AGA ACT GCA GGG TAT GGT GAG AA
#A CCA ACT ACG GAT TGC 390
TGC AAA CCA CAC CTT CTC TTT CTT ATG TCT TT
#T TAC TAC AAA CTA CAA 438
GAC AAT TGT TGA AAC CTG CTA TAC ATG TTT AT
#T TTA ATA AAT TGA TGG 486
CAA AAA CTG
#
#
# 495
(2) INFORMATION FOR SEQ ID NO: 5:
          (i) SEQUENCE CHARACTERISTICS:
                    (A) LENGTH: 111 amino
#acids
                    (B) TYPE: amino acid
                    (C) STRANDEDNESS: double
                    (D) TOPOLOGY: linear
        (ii) MOLECULE TYPE: <Unknown>
        (xi) SEQUENCE DESCRIPTION: SEQ ID NO:
#5:
Met Ser Thr Ile Lys Leu Ser Leu Cys Leu Le
#u Ile Met Leu Ala Val
1 5
# 10
# 15
Cys Cys Tyr Glu Ala Asn Ala Ser Gln Ile Cy
#s Glu Leu Val Ala His
                        20
# 25
# 30
Glu Thr Ile Ser Phe Leu Met Lys Ser Glu Gl
#u Glu Leu Lys Lys Glu
                35
# 40
# 45
Leu Glu Met Tyr Asn Ala Pro Pro Ala Ala Va
#l Glu Ala Lys Leu Glu
        50
# 55
# 60
Val Lys Arg Cys Val Asp Gln Met Ser Asn Gl
#y Asp Arg Leu Val Val
65
#70
#75
#80
Ala Glu Thr Leu Val Tyr Ile Phe Leu Glu Cy
#s Gly Val Lys Gln Trp
                                85
# 90
# 95
Val Glu Thr Tyr Tyr Pro Glu Ile Asp Phe Ty
#r Tyr Asp Met Asn
                        100
# 105
# 110
(2) INFORMATION FOR SEQ ID NO: 6:
          (i) SEQUENCE CHARACTERISTICS:
                    (A) LENGTH: 412 base
#pairs
                    (B) TYPE: nucleic acid
                    (C) STRANDEDNESS: double
                    (D) TOPOLOGY: linear
        (ii) MOLECULE TYPE: cDNA
        (xi) SEQUENCE DESCRIPTION: SEQ ID NO:
#6:
CGCTAAGTAG AAAACTGAA ATG AGC ACC ATT AAG CTG AGC
# CTG TGT CTT CTG 52
# Met Ser Thr Ile Lys Leu Ser Leu Cys
# Leu Leu
# 1
# 5
# 10
ATC ATG CTG GCT GTT TGT TGC TAT GAA GCT AA
#T GCT AGC CAG ATC TGT 100
Ile Met Leu Ala Val Cys Cys Tyr Glu Ala As
#n Ala Ser Gln Ile Cys
                          15
# 20
# 25
GAA CTT GTT GCC CAT GAA ACC ATA AGC TTC TT
#A ATG AAA AGT GAG GAA 148
Glu Leu Val Ala His Glu Thr Ile Ser Phe Le
#u Met Lys Ser Glu Glu
                  30
# 35
# 40
GAA CTG AAG AAG GAA CTT GAG ATG TAT AAT GC
#A CCT CCA GCA GCT GTT 196
Glu Leu Lys Lys Glu Leu Glu Met Tyr Asn Al
#a Pro Pro Ala Ala Val
          45
# 50
# 55
GAA GCA AAA CTG GAA GTG AAG AGA TGT GTA GA
#C CAG ATG AGC AAT GGA 244
Glu Ala Lys Leu Glu Val Lys Arg Cys Val As
#p Gln Met Ser Asn Gly
  60
# 65
# 70
# 75
GAC AGA TTG GTA GTA GCA GAA ACA CTG GTA TA
#C ATT TTT TTG GAA TGT 292
Asp Arg Leu Val Val Ala Glu Thr Leu Val Ty
#r Ile Phe Leu Glu Cys
                                  80
# 85
# 90
GGT GTG AAA CAA TGG GTA GAA ACA TAT TAT CC
#T GAG ATC GAT TTC TAC 340
Gly Val Lys Gln Trp Val Glu Thr Tyr Tyr Pr
#o Glu Ile Asp Phe Tyr
                          95
# 100
# 105
TAC GAT ATG AAC TGA TTT TTC CTG TTC AAT GT
#G ATG GTT TCA AGT CTT 388
Tyr Asp Met Asn *
                110
GCA CCA ATA AAT TAT TCT CCT TGC
#
# 412
(2) INFORMATION FOR SEQ ID NO: 7:
          (i) SEQUENCE CHARACTERISTICS:
                    (A) LENGTH: 440 base
#pairs
                    (B) TYPE: nucleic acid
                    (C) STRANDEDNESS: double
                    (D) TOPOLOGY: linear
        (ii) MOLECULE TYPE: cDNA
        (xi) SEQUENCE DESCRIPTION: SEQ ID NO:
#7:
GCTCATCCTT TGCTAAGTCT GAAAACAAAC TGAGCACCAT GAAGCTGTCC CT
#GTGTCTTC 60
TGTTGGTCAT CCTGGCTGTT CATTGCTATG AAGCTAATGC TGCAAACGTC TG
#TCCAGCAG 120
TTCTTTCTGT AAGCAAATCT TTCCTATTTG ACAAGGTGGA GAAATTTGAG GC
#CTATCTTC 180
AGACATTTAA CGCACCTCCA GAGGCTGTTA AAGCAAAAGT GGAAGTGAAG AA
#ATGTATAG 240
ACAGCACTCT GAACTATTTA GAGAAAATGG AAATGGGAAA AATACTGGCA GA
#AGTCGTTG 300
GGTATTGTAA AGGAACAGAA AACTGAAACA TGGCTCTTCC TGGTCTCCAT TG
#CTTCTCAC 360
AGATAAACTG ACTTTCCTTG CCCAATGTGA AGGTTTCAAC GTCTTGCACT AA
#TAAATTAC 420
TCTCCTTGCA TGTTAAAAAA
#
#
#440
(2) INFORMATION FOR SEQ ID NO: 8:
          (i) SEQUENCE CHARACTERISTICS:
                    (A) LENGTH: 112 amino
#acids
                    (B) TYPE: amino acid
                    (D) TOPOLOGY: unknown
        (ii) MOLECULE TYPE: protein
        (xi) SEQUENCE DESCRIPTION: SEQ ID NO:
#8:
Met Arg Leu Ser Leu Cys Leu Leu Thr Ile Le
#u Val Val Cys Cys Tyr
1 5
# 10
# 15
Glu Ala Asn Gly Gln Thr Leu Ala Gly Gln Va
#l Cys Gln Ala Leu Gln
                        20
# 25
# 30
Asp Val Thr Ile Thr Phe Leu Leu Asn Pro Gl
#u Glu Glu Leu Lys Arg
                35
# 40
# 45
Glu Leu Glu Glu Phe Asp Ala Pro Pro Glu Al
#a Val Glu Ala Asn Leu
        50
# 55
# 60
Lys Val Lys Arg Cys Ile Asn Lys Ile Met Ty
#r Gly Asp Arg Leu Ser
65
#70
#75
#80
Met Gly Thr Ser Leu Val Phe Ile Met Leu Ly
#s Cys Asp Val Lys Val
                                85
# 90
# 95
Trp Leu Gln Ile Asn Phe Pro Arg Gly Arg Tr
#p Phe Ser Glu Ile Asn
                        100
# 105
# 110
(2) INFORMATION FOR SEQ ID NO: 9:
          (i) SEQUENCE CHARACTERISTICS:
                    (A) LENGTH: 91 amino
#acids
                    (B) TYPE: amino acid
                    (D) TOPOLOGY: unknown
        (ii) MOLECULE TYPE: protein
        (xi) SEQUENCE DESCRIPTION: SEQ ID NO:
#9:
Met Lys Leu Ala Ile Thr Leu Ala Leu Val Th
#r Leu Ala Leu Leu Cys
1 5
# 10
# 15
Ser Pro Ala Ser Ala Gly Ile Cys Pro Arg Ph
#e Ala His Val Ile Glu
                        20
# 25
# 30
Asn Leu Leu Leu Gly Thr Pro Ser Ser Tyr Gl
#u Thr Ser Leu Lys Glu
                35
# 40
# 45
Phe Glu Pro Asp Asp Thr Met Lys Asp Ala Gl
#y Met Gln Met Lys Lys
        50
# 55
# 60
Val Leu Asp Ser Leu Pro Gln Thr Thr Arg Gl
#u Asn Ile Met Lys Leu
65
#70
#75
#80
Thr Glu Lys Ile Val Lys Ser Pro Leu Cys Me
#t
                                85
# 90
(2) INFORMATION FOR SEQ ID NO: 10:
          (i) SEQUENCE CHARACTERISTICS:
                    (A) LENGTH: 93 amino
#acids
                    (B) TYPE: amino acid
                    (C) STRANDEDNESS: double
                    (D) TOPOLOGY: linear
        (ii) MOLECULE TYPE: <Unknown>
        (xi) SEQUENCE DESCRIPTION: SEQ ID NO:
#10:
Met Lys Leu Leu Met Val Leu Met Leu Ala Al
#a Leu Ser Gln His Cys
1 5
# 10
# 15
Tyr Ala Gly Ser Gly Cys Pro Leu Leu Glu As
#n Val Ile Ser Lys Thr
                        20
# 25
# 30
Ile Asn Pro Gln Val Ser Lys Thr Glu Tyr Ly
#s Glu Leu Leu Gln Glu
                35
# 40
# 45
Phe Ile Asp Asp Asn Ala Thr Thr Asn Ala Il
#e Asp Glu Leu Lys Glu
        50
# 55
# 60
Cys Phe Leu Asn Gln Thr Asp Glu Thr Leu Se
#r Asn Val Glu Val Phe
65
#70
#75
#80
Met Gln Leu Ile Tyr Asp Ser Ser Leu Cys As
#p Leu Phe
                                85
# 90
(2) INFORMATION FOR SEQ ID NO: 11:
          (i) SEQUENCE CHARACTERISTICS:
                    (A) LENGTH: 503 base
#pairs
                    (B) TYPE: nucleic acid
                    (C) STRANDEDNESS: double
                    (D) TOPOLOGY: linear
        (ii) MOLECULE TYPE: cDNA
        (xi) SEQUENCE DESCRIPTION: SEQ ID NO:
#11:
GACAGCGGCT TCCTTGATCC TTGCCACCCG CGACTGAACA CCGACAGCAG CA
#GCCTCACC 60
ATG AAG TTG CTG ATG GTC CTC ATG CTG GCG GC
#C CTC TCC CAG CAC TGC 108
Met Lys Leu Leu Met Val Leu Met Leu Ala Al
#a Leu Ser Gln His Cys
    1 5
# 10
# 15
TAC GCA GGC TCT GGC TGC CCC TTA TTG GAG AA
#T GTG ATT TCC AAG ACA 156
Tyr Ala Gly Ser Gly Cys Pro Leu Leu Glu As
#n Val Ile Ser Lys Thr
                          20
# 25
# 30
ATC AAT CCA CAA GTG TCT AAG ACT GAA TAC AA
#A GAA CTT CTT CAA GAG 204
Ile Asn Pro Gln Val Ser Lys Thr Glu Tyr Ly
#s Glu Leu Leu Gln Glu
                  35
# 40
# 45
TTC ATA GAC GAC AAT GCC ACT ACA AAT GCC AT
#A GAT GAA TTG AAG GAA 252
Phe Ile Asp Asp Asn Ala Thr Thr Asn Ala Il
#e Asp Glu Leu Lys Glu
          50
# 55
# 60
TGT TTT CTT AAC CAA ACG GAT GAA ACT CTG AG
#C AAT GTT GAG GTG TTT 300
Cys Phe Leu Asn Gln Thr Asp Glu Thr Leu Se
#r Asn Val Glu Val Phe
  65
# 70
# 75
# 80
ATG CAA TTA ATA TAT GAC AGC AGT CTT TGT GA
#T TTA TTT TAA CTT TCT 348
Met Gln Leu Ile Tyr Asp Ser Ser Leu Cys As
#p Leu Phe *
                                  85
# 90
GCA AGA CCT TTG GCT CAC AGA ACT GCA GGG TA
#T GGT GAG AAA CCA ACT 396
ACG GAT TGC TGC AAA CCA CAC CTT CTC TTT CT
#T ATG TCT TTT TAC TAC 444
AAA CTA CAA GAC AAT TGT TGA AAC CTG CTA TA
#C ATG TTT ATT TTA ATA 492
AAT TGA TGG CA
#
#
# 503
(2) INFORMATION FOR SEQ ID NO: 12:
          (i) SEQUENCE CHARACTERISTICS:
                    (A) LENGTH: 95 amino
#acids
                    (B) TYPE: amino acid
                    (C) STRANDEDNESS: double
                    (D) TOPOLOGY: linear
        (ii) MOLECULE TYPE: <Unknown>
        (xi) SEQUENCE DESCRIPTION: SEQ ID NO:
#12:
Met Lys Leu Val Phe Leu Phe Leu Leu Val Th
#r Ile Pro Ile Cys Cys
1 5
# 10
# 15
Tyr Ala Ser Gly Ser Gly Cys Ser Ile Leu As
#p Glu Val Ile Arg Gly
                        20
# 25
# 30
Thr Ile Asn Ser Thr Val Thr Leu His Asp Ty
#r Met Lys Leu Val Lys
                35
# 40
# 45
Pro Tyr Val Gln Asp His Phe Thr Glu Lys Al
#a Val Lys Gln Phe Lys
        50
# 55
# 60
Gln Cys Phe Leu Asp Gln Thr Asp Lys Thr Le
#u Glu Asn Val Gly Val
65
#70
#75
#80
Met Met Glu Ala Ile Phe Asn Ser Glu Ser Cy
#s Gln Gln Pro Ser
                                85
# 90
# 95
(2) INFORMATION FOR SEQ ID NO: 13:
          (i) SEQUENCE CHARACTERISTICS:
                    (A) LENGTH: 509 base
#pairs
                    (B) TYPE: nucleic acid
                    (C) STRANDEDNESS: double
                    (D) TOPOLOGY: linear
        (ii) MOLECULE TYPE: cDNA
        (xi) SEQUENCE DESCRIPTION: SEQ ID NO:
#13:
AGTTTCCTGA TTTCTGTCTT GGACAACAGA ACAACCCACA GGGACTGCCT CA
#AC ATG 57
#
#
# Met
#
#
# 1
AAG CTG GTG TTT CTA TTC TTG TTG GTC ACC AT
#C CCT ATT TGC TGC TAT 105
Lys Leu Val Phe Leu Phe Leu Leu Val Thr Il
#e Pro Ile Cys Cys Tyr
                            5
# 10
# 15
GCC AGT GGT TCT GGC TGC AGT ATT CTA GAT GA
#A GTT ATT AGA GGT ACA 153
Ala Ser Gly Ser Gly Cys Ser Ile Leu Asp Gl
#u Val Ile Arg Gly Thr
                  20
# 25
# 30
ATT AAC TCA ACT GTG ACT TTA CAT GAC TAT AT
#G AAA TTA GTT AAG CCA 201
Ile Asn Ser Thr Val Thr Leu His Asp Tyr Me
#t Lys Leu Val Lys Pro
          35
# 40
# 45
TAT GTA CAA GAT CAT TTT ACT GAA AAG GCT GT
#G AAG CAA TTC AAG CAG 249
Tyr Val Gln Asp His Phe Thr Glu Lys Ala Va
#l Lys Gln Phe Lys Gln
  50
# 55
# 60
# 65
TGT TTT CTA GAT CAG ACC GAC AAG ACT CTG GA
#A AAT GTT GGC GTG ATG 297
Cys Phe Leu Asp Gln Thr Asp Lys Thr Leu Gl
#u Asn Val Gly Val Met
                                  70
# 75
# 80
ATG GAG GCA ATA TTT AAC AGT GAA AGC TGT CA
#A CAG CCA TCC TAA ACA 345
Met Glu Ala Ile Phe Asn Ser Glu Ser Cys Gl
#n Gln Pro Ser *
                          85
# 90
# 95
TCT ACA AGA TCT TTG GCC ACA GGA CTC CAG GA
#A ACT GGC AAT GGC CAA 393
GCA ACT GAT AAC ACA GAT CAT AAC TCT TCT TT
#C TTG AAC CCC TTT TTC 441
TAC CTA TAA AGT GCA AGA CGA TTG TTG AAA CC
#T CAA ATT TAT GTC TTT 489
CCA TTT TAT TAA ATT ATC TG
#
#
#509
```

What is claimed is:

1. An isolated polypeptide comprising an amino acid sequence selected from the group consisting of:
a) a polypeptide comprising an amino acid sequence selected from the group consisting of SEQ ID NO:I and SEQ ID NO:3,
b) a naturally occurring polypeptide comprising an amino acid sequence at least 90% identical to an amino acid sequence selected from the group consisting of SEQ ID NO:1 and SEQ ID NO:3,
c) a biologically active fragment of a polypeptide having an amino acid sequence selected from the group consisting of SEQ ID NO:1 and SEQ ID NO:3, and
d) an immunogenic fragment of a polypeptide having an amino acid sequence selected from the group consisting of SEQ ID NO:1 and SEQ ID NO:3.

2. An isolated polypeptide of claim 1, having a sequence selected from the group consisting of SEQ ID NO:1 and SEQ ID NO:3.

3. An isolated polynucleotide encoding a polypeptide of claim 1.

4. An isolated polynucleotide encoding a polypeptide of claim 2.

5. An isolated polynucleotide of claim 4, having a sequence selected from the group consisting of SEQ ID NO:2 and SEQ ID NO:4.

6. A recombinant polynucleotide comprising a promoter sequence operably linked to a polynucleotide of claim 3.

7. A cell transformed with a recombinant polynucleotide of claim 6.

8. A transgenic organism comprising a recombinant polynucleotide of claim 6.

9. A method for producing a polypeptide of claim 1, the method comprising:
a) culturing a cell under conditions suitable for expression of the polypeptide, wherein
said cell is transformed with a recombinant polynucleotide, and said recombinant polynucleotide comprises a promoter sequence operably linked to a polynucleotide encoding the polypeptide of claim 1, and
b) recovering the polypeptide so expressed.

10. A method of claim 9, wherein the polypeptide has the sequence selected from the group consisting of SEQ ID NO:1 and SEQ ID NO:3.

11. An isolated antibody which specifically binds to a polypeptide of claim 1.

12. An isolated polynucleotide comprising a sequence selected from the group consisting of:
a) a polynucleotide comprising a polynucleotide sequence selected from the group consisting of SEQ ID NO:2 and SEQ ID NO:4,
b) a naturally occurring polynucleotide comprising a polynucleotide sequence at least 90% identical to a polynucleotide sequence selected from the group consisting of SEQ ID NO:2 and SEQ ID NO:4,
c) a polynucleotide having a sequence complementary to a polynucleotide of a),
d) a polynucleotide having a sequence complementary to a polynucleotide of b) and
e) an RNA equivalent of a)-d).

13. An isolated polynucleotide comprising at least 60 contiguous nucleotides of a polynucleotide of claim 12.

14. A method for detecting a target polynucleotide in a sample, said target polynucleotide having a sequence of a polynucleotide of claim 12, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

15. A method of claim 14, wherein the probe comprises at least 60 contiguous nucleotides.

16. A method for detecting a target polynucleotide in a sample, said target polynucleotide having a sequence of a polynucleotide of claim 12, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

17. A composition comprising a polypeptide of claim 1 and a pharmaceutically acceptable excipient.

18. A composition of claim 17, wherein the polypeptide has an amino acid sequence selected from the group consisting of SEQ ID NO:1 and SEQ ID NO:3.

19. A method for treating a disease or condition associated with decreased expression of functional hSBP, comprising administering to a patient in need of such treatment the composition of claim 17.

20. A method for screening a compound for effectiveness as an agonist of a polypeptide of claim 1, the method comprising:
a) exposing a sample comprising a polypeptide of claim 1 to a compound, and
b) detecting agonist activity in the sample.

21. A composition comprising an agonist compound identified by a method of claim 20 and a pharmaceutically acceptable excipient.

22. A method for treating a disease or condition associated with decreased expression of functional hSBP, comprising administering to a patient in need of such treatment a composition of claim 21.

23. A method for screening a compound for effectiveness as an antagonist of a polypeptide of claim 1, the method comprising:
a) exposing a sample comprising a polypeptide of claim 1 to a compound, and
b) detecting antagonist activity in the sample.

24. A composition comprising an antagonist compound identified by a method of claim 23 and a pharmaceutically acceptable excipient.

25. A method for treating a disease or condition associated with overexpression of functional hSBP, comprising administering to a patient in need of such treatment a composition of claim 24.

26. A method of screening for a compound that specifically binds to the polypeptide of claim 1, said method comprising the steps of:
a) combining the polypeptide of claim 1 with at least one test compound under suitable conditions, and
b) detecting binding of the polypeptide of claim 1 to the test compound, thereby identifying a compound that specifically binds to the polypeptide of claim 1.

27. A method of screening for a compound that modulates the activity of the polypeptide of claim 1, said method comprising:
a) combining the polypeptide of claim 1 with at least one test compound under conditions permissive for the activity of the polypeptide of claim 1,
b) assessing the activity of the polypeptide of claim 1 in the presence of the test compound, and
c) comparing the activity of the polypeptide of claim 1 in the presence of the test compound with the activity of the polypeptide of claim 1 in the absence of the test compound, wherein a change in the activity of the polypeptide of claim 1 in the presence of the test compound is indicative of a compound that modulates the activity of the polypeptide of claim 1.

28. A method for screening a compound for effectiveness in altering expression of a target polynucleotide, wherein said target polynucleotide comprises a polynucleotide sequence of claim 5, the method comprising:
a) exposing a sample comprising the target polynucleotide to a compound, under conditions suitable for the expression of the target polynucleotide,
b) detecting altered expression of the target polynucleotide, and
c) comparing the expression of the target polynucleotide in the presence of varying amounts of the compound and in the absence of the compound.

29. A method for assessing toxicity of a test compound, said method comprising:
a) treating a biological sample containing nucleic acids with the test compound;
b) hybridizing the nucleic acids of the treated biological sample with a probe comprising at least 20 contiguous nucleotides of a polynucleotide of claim 12 under conditions whereby a specific hybridization complex is formed between said probe and a target polynucleotide in the biological sample, said target polynucleotide comprising a polynucleotide sequence of a polynucleotide of claim 12 or fragment thereof;
c) quantifying the amount of hybridization complex; and
d) comparing the amount of hybridization complex in the treated biological sample with the amount of hybridization complex in an un treated biological sample, wherein a difference in the amount of hybridization complex in the treated biological sample is indicative of toxicity of the test compound.

30. A diagnostic test for a condition or disease associated with the expression of hSBP in a biological sample comprising the steps of:
a) combining the biological sample with an antibody of claim 11, under conditions suitable for the antibody to bind the polypeptide and form an antibody:polypeptide complex; and
b) detecting the complex, wherein the presence of the complex correlates with the presence of the polypeptide in the biological sample.

31. The antibody of claim 11, wherein the antibody is:
a) a chimeric antibody,
b) a single chain antibody,
c) a Fab fragment,
d) a F(ab′)₂ fragment, or
e) a humanized antibody.

32. A composition comprising an antibody of claim 11 and an acceptable excipient.

33. A method of diagnosing a condition or disease associated with the expression of hSBP in a subject, comprising administering to said subject an effective amount of the composition of claim 32.

34. A composition of claim 32, wherein the antibody is labeled.

35. A method of diagnosing a condition or disease associated with the expression of hSBP in a subject, comprising administering to said subject an effective amount of the composition of claim 34.

36. A method of preparing a polyclonal antibody with the specificity of the antibody of claim 11 comprising:
a) immunizing an animal with a polypeptide having an amino acid sequence selected from the group consisting of SEQ ID NO:1 and SEQ ID NO:3 or an immunogenic fragment thereof, under conditions to elicit an antibody response;
b) isolating antibodies from said animal; and
c) screening the isolated antibodies with the polypeptide, thereby identifying a polyclonal antibody which binds specifically to a polypeptide having an amino acid sequence selected from the group consisting of SEQ ID NO:1 and SEQ ID NO:3.

37. An antibody produced by a method of claim 36.

38. A composition comprising the antibody of claim 37 and a suitable carrier.

39. A method of making a monoclonal antibody with the specificity of the antibody of claim 11 comprising:
a) immunizing an animal with a polypeptide having an amino acid sequence selected from the group consisting of SEQ ID NO:1 and SEQ ID NO:3, or an immunogenic fragment thereof, under conditions to elicit an antibody response;
b) isolating antibody producing cells from the animal;
c) fusing the antibody producing cells with immortalized cells to form monoclonal antibody-producing hybridoma cells;
d) culturing the hybridoma cells; and
e) isolating from the culture monoclonal antibody which binds specifically to a polypeptide having an amino acid sequence selected from the group consisting of SEQ ID NO: I and SEQ ID NO:3.

40. A monoclonal antibody produced by a method of claim 39.

41. A composition comprising the antibody of claim 40 and a suitable carrier.

42. The antibody of claim 11, wherein the antibody is produced by screening a Fab expression library.

43. The antibody of claim 11, wherein the antibody is produced by screening a recombinant immunoglobulin library.

44. A method for detecting a polypeptide having an amino acid sequence selected from the group consisting of SEQ ID NO:1 and SEQ ID NO:3 in a sample, comprising the steps of:
a) incubating the antibody of claim 11 with a sample under conditions to allow specific binding of the antibody and the polypeptide; and
b) detecting specific binding, wherein specific binding indicates the presence of a polypeptide having an amino acid sequence selected from the group consisting of SEQ ID NO:1 and SEQ ID NO:3 in the sample.

45. A method of purifying a polypeptide having an amino acid sequence selected from the group consisting of SEQ ID NO:1 and SEQ ID NO:3 from a sample, the method comprising:
a) incubating the antibody of claim 11 with a sample under conditions to allow specific binding of the antibody and the polypeptide; and
b) separating the antibody from the sample and obtaining the purified polypeptide having an amino acid sequence selected from the group consisting of SEQ ID NO:1 and SEQ ID NO:3.

46. A microarray wherein at least one element of the microarray is a polynucleotide of claim 13.

47. A method for generating a transcript image of a sample which contains polynucleotides, the method comprising the steps of:
a) labeling the polynucleotides of the sample,
b) contacting the elements of the microarray of claim 46 with the labeled polynucleotides of the sample under conditions suitable for the formation of a hybridization complex, and
c) quantifying the expression of the polynucleotides in the sample.

48. An array comprising different nucleotide molecules affixed in distinct physical locations on a solid substrate, wherein at least one of said nucleotide molecules comprises a first oligonucleotide or polynucleotide sequence specifically hybridizable with at least 30 contiguous nucleotides of a target polynucleotide, said target polynucleotide having a sequence of claim 12.

49. An array of claim 48, wherein said first oligonucleotide or polynucleotide sequence is completely complementary to at least 30 contiguous nucleotides of said target polynucleotide.

50. An array of claim 48, wherein said first oligonucleotide or polynucleotide sequence is completely complementary to at least 60 contiguous nucleotides of said target polynucleotide.

51. An array of claim 48, which is a microarray.

52. An array of claim 48, further comprising said target polynucleotide hybridized to said first oligonucleotide or polynucleotide.

53. An array of claim 48, wherein a linker joins at least one of said nucleotide molecules to said solid substrate.

54. An array of claim 48, wherein each distinct physical location on the substrate contains multiple nucleotide molecules having the same sequence, and each distinct physical location on the substrate contains nucleotide molecules having a sequence which differs from the sequence of nucleotide molecules at another physical location on the substrate.

55. A polypeptide of claim 1, comprising the amino acid sequence of SEQ ID NO:1.

56. A polypeptide of claim 1, comprising the amino acid sequence of SEQ ID NO:3.

57. A polynucleotide of claim 12, comprising the polynucleotide sequence of SEQ ID NO:2.

58. A polynucleotide of claim 12, comprising the polynucleotide sequence of SEQ ID NO:4.
